(19) United States
(12) Patent Application Publication
(10) Pub. No.: US 2008/0138898 A1
(43) Pub. Date: Jun. 12, 2008

(54) METHODS FOR IMPROVING ANTIBODY PRODUCTION

(75) Inventors: Xiao-Mai ZHOU, Watertown, MA (US); Daniel Tavares, Natick, MA (US)

Correspondence Address: SUGHRUE MION, PLLC, 2100 PENNSYLVANIA AVENUE, N.W., SUITE 800, WASHINGTON, DC, 20037, US

(73) Assignee: IMMUNOGEN, INC., Cambridge, MA (US)

(21) Appl. No.: 11/929,334

(22) Filed: Oct. 30, 2007

Related U.S. Application Data
(60) Provisional application No. 60/855,361, filed on Oct. 31, 2006.

(51) Int. Cl. C12N 5/02; C07H 21/02; C07K 16/00

(52) U.S. Cl. 435/375; 536/23.1; 530/387.1

(57) ABSTRACT

The present invention encompasses manufacturing of antibody variants, such as variant of huC242, or fragments thereof, wherein the variants are manufactured by substituting one or more amino acid residues in a parent antibody. Such substitution(s) is preferably done in a variable region framework sequence of the parent antibody comprising a heavy and a light chain. As a consequence of such substitution(s), variant antibodies or fragments thereof show enhanced antibody synthesis when introduced in a host cell as compared to the parent antibody.

This application claims priority to U.S. Provisional Application No. 60/855,361, filed Oct. 31, 2006, the entire disclosure of which is incorporated herein by reference.

### FIELD OF INVENTION

The present invention is directed to methods of improving antibody production. More particularly, to methods wherein antibodies are reengineered such that the reengineered antibodies are produced in a greater yield in host cells as compared to the parent antibody of the reengineered antibody.

### BACKGROUND

Monoclonal antibodies have a wide range of uses including in vitro diagnostics, laboratory reagents, and therapeutics. Currently there are at least 200 antibodies or antibody fragments undergoing clinical trials (Morrow, K. J., Jr., Monoclonal antibody production techniques. Gen. Eng. News, 2002, 20(14): 21).

High level expression of antibodies in CHO cells requires optimal efficiency from transcription through translation and secretion. Mammalian expression plasmids are primarily designed to achieve high mRNA levels through the use of potent viral enhancers like the hCMV immediate early gene enhancer and promoter sequence together with transcript-stabilizing polyadenylation signals like the SV40 poly A site. Synthetic cDNA constructs can be designed to further enhance mRNA levels by eliminating cryptic splice sites and other potentially detrimental cis elements within the antibody coding sequence. In addition, the synthetic constructs may enhance the gene translation machinery through optimal codon usage and by minimizing mRNA secondary structure energies (Trinh R, Gurbaxani B, Morrison S L, Seyfzadeh M. Optimization of codon pair use within the (GGGGS)3 linker sequence results in enhanced protein expression. Mol. Immunol. 2004 January; 40(10):717-22). However even with such optimized expression systems, expression levels in mammalian cells can vary significantly among different antibodies. Analysis of the different cellular steps that are necessary for the synthesis of antibody molecules from expression plasmids, led to the notion that properties of the variable region of an antibody can affect the levels of expression of a given antibody. However little is known about the characteristics of the sequence and structure of variable regions that can affect gene expression irrespective of transcription or translation efficiency.

Many antibodies derived from specific subclasses of variable region genes are biophysically predisposed to poor stability that may lead to low gene expression. Human light and heavy chain variable regions can be classified into subgroups with varying degrees of structural stability based on an analysis of a human scFv phage library (Ewert_S. Honegger A, Plückthun A. Structure-based improvement of the biophysical properties of immunoglobulin VH domains with a generalizable approach. Biochemistry. 2003 Feb. 18; 42(6):1517-28). An antibody or fragment belonging to a subgroup with members of poor stability may have a propensity to aggregate and may be difficult to express due to inefficient folding or assembly.

Further, residues that play critical roles in processes like folding and secretion are often highly conserved in germ line sequences, but may be altered during generation of the primary antibody repertoire and affinity maturation through somatic mutations. This can lead to antibodies having poor stability and low expression. A single residue change can dramatically alter chaperone binding or light chain/heavy chain assembly and result in an increase in intracellular unpaired heavy chain that is eventually degraded (Dul J L, Argon Y. A single amino acid substitution in the variable region of the light chain specifically blocks immunoglobulin secretion. Proc Natl Acad Sci USA. 1990 October; 87(20):8135-9; Wiens G D, Lekkerkerker A, Veltman I, Rittenberg M B. Mutation of a single conserved residue in VH complementarity-determining region 2 results in a severe Ig secretion defect. J. Immunol. 2001 Aug. 15; 167(4):2179-86). Other destabilizing mutations include the introduction of buried hydrophilic residues or surface hydrophobic residues. Invariant core packing residues like the heavy chain Glu6/Gln6 are also sensitive to residue changes in neighboring positions like H7 and H10 (Honegger A, Plückthun A. The influence of the buried glutamine or glutamate residue in position 6 on the structure of immunoglobulin variable domains. J Mol. Biol. 2001 Jun. 8; 309(3):687-99). As long as a somatic mutation does not cross a critical structural threshold, many biophysically undesirable sequences can be found in naturally occurring antibodies.

The stability and expression potential of many antibodies can be enhanced with a rational sequence reengineering approach. One of the simplest approaches to reengineering an antibody is to utilize the information present in data bases of thousands of antibody sequences (see e.g. Johnson G, Wu T T. Kabat Database and its applications: future directions. Nucleic Acids Res. 2001 Jan. 1; 29(1):205-6.) Careful analysis might identify potentially problematic residues. For example, individual residues that are rarely found in a given position can be altered to match a consensus residue for this position to improve stability (Steipe B, Schiller B, Plückthun A, Steinbacher S. Sequence statistics reliably predict stabilizing mutations in a protein domain. J Mol. Biol. 1994 Jul. 15; 240(3):188-92.) and even expression in mammalian cell lines (Whitcomb E A, Martin T M, Rittenberg M B. Restoration of Ig secretion: mutation of germline-encoded residues in T15L chains leads to secretion of free light chains and assembled antibody complexes bearing secretion-impaired heavy chains. J. Immunol. 2003 Feb. 15; 170(4):1903-9). The identification and reversal of biophysically offensive residues such as hydrophobic surface residues can also lead to improved expression (Nieba L, Honegger A, Krebber C, Plückthun A. Disrupting the hydrophobic patches at the antibody variable/constant domain interface: improved in vivo folding and physical characterization of an engineered scFv fragment. Protein Eng. 1997 April; 10(4):435-44). The majority of the data presently available to support the rational redesign of biophysically stable antibodies have been generated with antibody fragments in bacterially expressed phage display systems (Ewert S, Honegger A, Plückthun A. Stability improvement of antibodies for extracellular and intracellular applications: CDR grafting to stable frameworks and structure-based framework engineering. Methods. 2004 October; 34(2):184-99. Review).

Humanization by CDR grafting techniques can either fix or avoid these stability issues by simply choosing a human donor variable region framework from one of the more stable germ line subgroups whenever possible (Ewert et al., 2004, supra). WO 2004/065417 provides a further improvement for producing such antibodies and/or antigen binding fragments in mammalian cell cultures at higher yields by comparing the Hypervariable region 1 (HVR1) and/or Hypervariable region 2 (HVR2) amino acid sequence of the variable domain of an antibody to a corresponding HVR1 and/or HVR2 amino acid sequence of each of human variable domain subgroup consensus amino acid sequences and selecting the subgroup consensus sequence that has the most sequence identity with the HVR1 and/or HVR2 amino acid sequence of the variable domain. In WO2004/065417, the consensus sequence is derived from antibodies with the most identical HVR1 and/or HVR2 and is applied to CDR-grafted antibodies where all the framework sequences are complete human sequences.

Other humanization methods, such as the methods of resurfacing of rodent antibodies (U.S. Pat. No. 5,639,641; Roguska M A, Pedersen J T, Keddy C A, Henry A H, Searle S J, Lambert J M, Goldmacher V S, Blättler W A, Rees A R, Guild B C. Humanization of murine monoclonal antibodies through variable domain resurfacing. Proc Natl Acad Sci USA. 1994 Feb. 1; 91(3):969-73; Pedersen J T, Henry A H, Searle S J, Guild B C, Roguska M, Rees A R. Comparison of surface accessible residues in human and murine immunoglobulin Fv domains. Implication for humanization of murine antibodies. J Mol. Biol. 1994 Jan. 21; 235(3):959-73), of veneering of antibodies (U.S. Pat. No. 6,797,492; Padlan, E. A. 1991, Mol. Immunolgy. 28:489-498), and deimmunizing of antibodies (publication No. WO98/52976) maintain the hydrophobic core of the murine variable region. As a consequence, such humanized antibodies with murine core structures in the variable region derived from a murine germ line with poor biophysical properties will likely inherit these properties. There is therefore a need for methods that can improve the biophysical properties of such humanized antibodies. These methods should yield higher expression of resurfaced antibodies from mammalian cells.

### SUMMARY OF THE INVENTION

The present invention provides in general a method to improve the biophysical properties of an antibody (hereinafter “parent antibody”) that results in increased antibody production. The method identifies one or more non-consensus amino acid residues in the variable region framework of the parent antibody and preferably replaces them with one or more consensus residues. Optionally, one or more amino acids may be replaced with a non-consensus residue for biophysical considerations.

The consensus residues are identified by aligning a collection of antibody variable region framework sequences from antibodies from the same species (e.g., murine) or across the species from the same genus (e.g., mus and rattus) or from across the genus or other taxonomic classification according to their presumed natural relationships as that to which the antibody from which the parent was derived belongs.

More particularly, the present invention encompasses a method for increasing production of a parent antibody or an epitope binding fragment thereof in a host cell by sequence reengineering. The sequence reengineering comprises:

a) aligning a collection of antibody variable region framework sequences from antibodies from the same species (e.g., murine) or across the species from the same genus (e.g., mus and rattus) or from across the genus or other taxonomic classification according to their presumed natural relationships as that to which the antibody from which the parent was derived belongs, wherein such alignment identifies amino acid residues most frequently found (consensus residues) at each position in the framework;

b) comparing the consensus residues with the corresponding residues in the parent antibody variable region framework sequence;

c) identifying in the parent antibody one or more non-consensus amino acid residues in the variable region framework sequence; and

d) substituting in the parent antibody or fragment thereof one or more non-consensus amino acid residues with the consensus residue at the equivalent position to produce a variant antibody, wherein the variant antibody is produced in the host cell at a higher yield as compared to the parent antibody.

Optionally, one or more amino acids may be replaced with a non-consensus residue for biophysical considerations.

The present invention also provides in general a method to improve the biophysical properties of a humanized antibody that results in increased antibody production. The method identifies one or more non-consensus amino acid residues in the core of the variable region framework of the humanized antibody and replaces them with one or more consensus residues. Optionally, one or more amino acids may be replaced with a non-consensus residue for the biophysical considerations. The consensus residues are identified by aligning a collection of antibody variable region framework sequences from antibodies from the same species or across the species (e.g., murine) from the same genus (e.g., mus and rattus) or from across the genus or other taxonomic classification according to their presumed natural relationships as that to which the antibody from which the parent was derived belongs.

Thus, the present invention encompasses a method for increasing production of a humanized antibody or an epitope binding fragment thereof in a host cell by sequence reengineering. The sequence engineering comprises:

a) aligning a collection of antibody variable region framework sequences from antibodies from the same species (e.g., murine) or across the species from the same genus (e.g., mus and rattus) or from across the genus or other taxonomic classification according to their presumed natural relationships, as that to which the humanized antibody was derived belongs, wherein such alignment identifies amino acid residues most frequently found (consensus residues) at each position in the framework;

b) comparing the consensus residues with the corresponding residues in the humanized antibody variable region framework sequence;

c) identifying in the humanized antibody one or more non-consensus residues in the variable region framework sequence; and

d) substituting in the humanized antibody or a fragment thereof said one or more non-consensus residues with the consensus residue at the equivalent position to produce a variant antibody, wherein the variant antibody is produced in a cell at a higher yield as compared to the humanized antibody.

Optionally, one or more amino acids may be replaced with a non-consensus residue for biophysical considerations.

In another aspect, the present invention provides a method to improve the biophysical properties of a humanized murine antibody that results in increased antibody production. The method identifies one or more non-consensus amino acid residues in the variable region framework of the humanized antibody and replaces them with one or more consensus residues. Optionally, one or more amino acids may be replaced with a non-consensus residue for the biophysical considerations. The consensus residues are identified by aligning a collection of antibody variable region framework sequences from murine antibodies.

More particularly, the present invention encompasses a method for increasing production of a humanized murine antibody or an epitope binding fragment thereof in a host cell by sequence reengineering. The sequence reengineering comprises:

a) aligning a collection of murine antibody variable region framework sequences, wherein such alignment identifies amino acid residues most frequently found (consensus residues) at each position in the framework;

b) comparing the consensus residues with the corresponding residues in the humanized antibody variable region framework sequence;

c) identifying in the humanized antibody one or more non-consensus amino acid residues in the variable region framework sequence; and

d) substituting in the variable region framework sequence of the humanized antibody or a fragment thereof said one or more non-consensus residues with the consensus residue at the equivalent position to produce a variant antibody, wherein the variant antibody is produced in a cell at a higher yield as compared to the humanized antibody.

Optionally, one or more amino acids may be replaced with a non-consensus residue for the biophysical considerations.

The invention also encompasses an isolated nucleic acids that encode the variant antibodies.

### BRIEF DESCRIPTION OF THE FIGURES

FIG. 1 shows a schematic representation of an IgG antibody and the variable regions of a heavy chain and a light chain. A cartoon representation of the heavy and light chain variable regions is shown to the right with the framework residues in grey and the CDR's in black. The Kabat antibody residue sequence numbers are given for the variable region endpoints as well as the boundaries for each CDR.

FIG. 2 shows the low huC242 production by 293T cells a few hours after transient transfection with a plasmid containing the huC242 genes. Plasmids for humanized antibodies A, B, and huC242 were normalized in concentration and introduced into 293T cells in parallel at 2 μg/ml. Secreted antibodies were collected from the culture medium at 14 hr, 22 hr and 48 hr after transfection. Antibody concentrations were determined using an anti-huIgG1 ELISA.

FIG. 3 shows huC242 HC and LC mRNA levels in transiently transfected 293T cells. HuC242 and other resurfaced antibodies A, B, C, D, E, F, were introduced into 293T cells in parallel. Total mRNA was isolated from each transfected cell sample 72 hrs after transfection and samples were subsequently reverse transcribed into cDNA.

FIG. 4 shows a gel with bands for assembled intact antibody, labeled H2L2, and heavy chain, labeled H from CHO cells that produce either huC242 or resurfaced Ab A. Expression and assembly of Ab. A and huC242 clone 1 and clone 2 were compared. The CHO cell lines for Ab. A and the two C242 clones were cultured in parallel and cells lysed. Whole cell lysates were subjected to protein A purification. Isolated IgGs were separated on a non-denaturing gel and stained with Coomassie Blue.

FIG. 5 A shows the heavy chain variable region sequence of the huC242 (SEQ ID NO:1) antibody aligned with the respective consensus sequence of murine antibodies in the Kabat database (SEQ ID NO:3). The CDR's are underlined and marked in bold. The residues that differ between the sequences are highlighted with grey backgrounds and the preferred residues discussed in detail herein are highlighted with black backgrounds. Surface residues are marked beneath with an asterisk “*”.

FIG. 5 B shows the light chain variable region sequence of the huC242 (SEQ ID NO:2) antibody aligned with the respective consensus sequence of murine antibodies in the Kabat database (SEQ ID NO:4). The CDR's are underlined and marked in bold. The residues that differ between the sequences are highlighted with grey backgrounds and the preferred residues discussed in detail in this patent are highlighted with black backgrounds. Surface residues are marked beneath with an asterisk “*”

FIG. 5 C shows alignment of light chain variable region sequence of the huC242 (SEQ ID NO:5) antibody aligned with respective consensus sequence of four resurfaced humanized murine antibodies from ImmunoGen (huMy96 LC, SEQ ID NO:6; rB4 LC, SEQ ID NO:7; huEM164 LC, SEQ ID NO:8; huN901 LC, SEQ ID NO:9; Consensus, SEQ ID NO:10), which shows that in the four humanized antibodies amino acid R is the conserved amino acid residue for the non-consensus Q found in the huC242. In the murine database R is replaced with K, which is the most conserved amino acid residue. In this case, K is replaceable with R because of similar properties of the two amino acids. Nevertheless, replacement of Q with K is encompassed to be within the scope of this invention. The alignment is based on Kabat.

FIG. 6 shows the moderate increase in IgG production caused by a single amino acid substitution in either the huC242 HC or LC framework. The productivity of huC242 variants with single framework amino acid substitutions are compared with that of the parent huC242 and antibody B. Equal amounts of plasmids were transfected into 293T cells. After 72 hr, levels of secreted IgG were determined with an ELISA. The binding of variant huC242 to the antigen-expressing Colo 205 cells was measured by FACS.

FIG. 7 shows the significant increase in IgG production by the combination of two or three huC242 HC and LC variations in 293T transient expression experiments. Original huC242 productivity is set as 1.0. Secreted IgGs were collected from culture medium 72 hr after transfection.

FIG. 8 shows that the mRNA levels of huC242 HC and LC variants remain unchanged. Specific variant huC242 mRNA levels were determined by qPCR, and normalized to neo mRNA.

FIG. 9 shows the increased accumulation of intracellular LC as a result of HC framework residue substitutions by whole cell lysate electrophoresis on a denaturing gel. 293T cells were lysed 72 hr after transfection. HC and LC were detected with anti-huIgG1 and anti-huK antibodies, respectively.

FIGS. 10(a) and 10(b) show that huC242 variations lead to increased HC and LC synthesis and increased assembly of whole antibody (H2L2) in cells.

FIG. 10(a): 293T cells were lysed 72 hr after transfection. The lysates were separated on a gel and transferred onto a membrane, which was probed for assembled and un-assembled IgG HC and LC (electrophoresis was under non-denaturing conditions). The blot was stripped and re-probed with anti-tubulin antibody to show sample loading levels.

FIG. 10(b): IgGs were isolated from the cell lysates prepared as described in FIG. 10(a) using protein A affinity beads. The isolated samples were then subjected to electrophoresis on a non-denaturing gel, which was subsequently stained with Coomassie Blue.

FIG. 11(a) shows a FACS analysis of the binding of huC242 and variants of huC242 to Colo 205 cells. Ab B is a non-binding control antibody.

FIG. 11(b) shows a FACS analysis of the binding of DM4 conjugates of huC242 and variants of huC242 to Colo 205 cells. Ab B is a non-binding control antibody.

FIG. 11(c) shows the results of competition binding of parent huC242 and variant huC242 antibodies with FITC-labeled parent huC242 on Colo 205 cells using FACS analysis. Antibody B serves as a non-binding, non-competing control.

FIG. 12 shows the huC242 amino acid and the nucleic acid sequences for the heavy chain (Panel A; SEQ ID NOs:11 and 12) and the light chain (Panel B; SEQ ID NOs:13 and 14). It also shows, in Panel C, the heavy chain variable domain sequence (SEQ ID NO:15) and the light chain variable domain sequence (SEQ ID NO:16) of the huC242 depicting the codon(s) that encode the amino acid changes identified in huC242.

### DETAILED DESCRIPTION OF THE INVENTION

While the invention is described with reference to humanized murine antibodies, those of ordinary skill in the art will understand that the reengineering method can be applied to any antibody for which there is a sufficiently large data base from which to derive a consensus sequence of the heavy chain and/or light chain variable region(s).

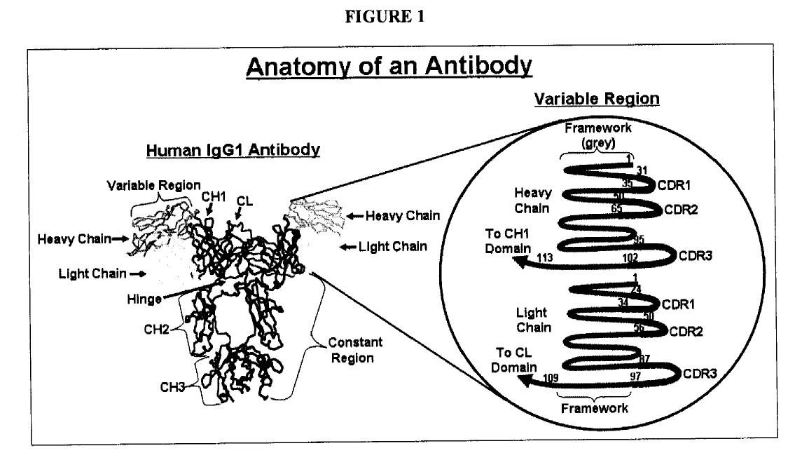

A standard way of generating monoclonal antibodies to human antigens is to immunize another animal species with the antigen, generate hybridomas with the immune B-cell of the animal, and select the hybridoma clones that secret antibodies that bind to the human antigen. Most commonly the animals used are mice or rats, thus the antibodies generated are murine antibodies. Monoclonal antibodies to human antigens are used in humans for diagnostic purposes or for the treatment of various diseases, such as cancer, autoimmune diseases, inflammation, and infections. However the use of murine monoclonal antibodies in humans is limited, because the antibodies are recognized as foreign proteins and elicit an immune response, often called a HAMA response (human anti-mouse antibody response). To prevent a HAMA response, methods have been developed for the humanization of murine antibodies. All methods replace the murine constant region domain (for the domain structure of an IgG see FIG. 1) with a human constant region domain, but differ in the humanization strategy for the variable region domain of the antibody. The method of CDR grafting transfers the six CDR domains from the murine variable region to a homologous human variable region by replacing the human CDR domains, thus the murine variable domain framework regions are entirely replaced by homologous human framework regions. Other humanization methods, such as the methods of resurfacing of rodent antibodies (U.S. Pat. No. 5,639,641; Roguska et al. 1994, Proc. Natl. Acad, Sci. USA 91:969-973, supra; Pedersen et al. 1994, J. Mol. Biol. 235:969-973, supra), of veneering of antibodies (U.S. Pat. No. 6,797,492; Padlan, E. A. 1991, Mol. Immunolgy. 28:489-498, supra), and deimmunizing of antibodies (international publication No. WO98/52976) maintain the hydrophobic core of the murine variable domain framework region and change only the surface exposed residues in the framework region with human residues. For example, an antibody humanized using the resurfacing technique contains human residues in every solvent accessible variable region framework position while preserving the murine residues in the CDR's and buried variable region framework positions. These humanized antibodies retain the binding affinity of the original murine antibody which is often lost when the hydrophobic core is replaced in other humanization methods, such as CDR grafting.

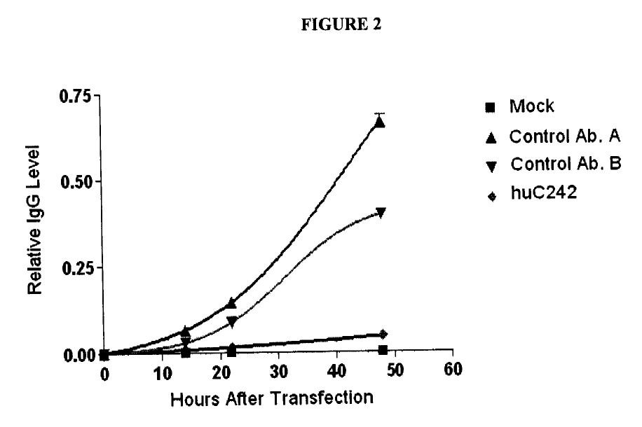

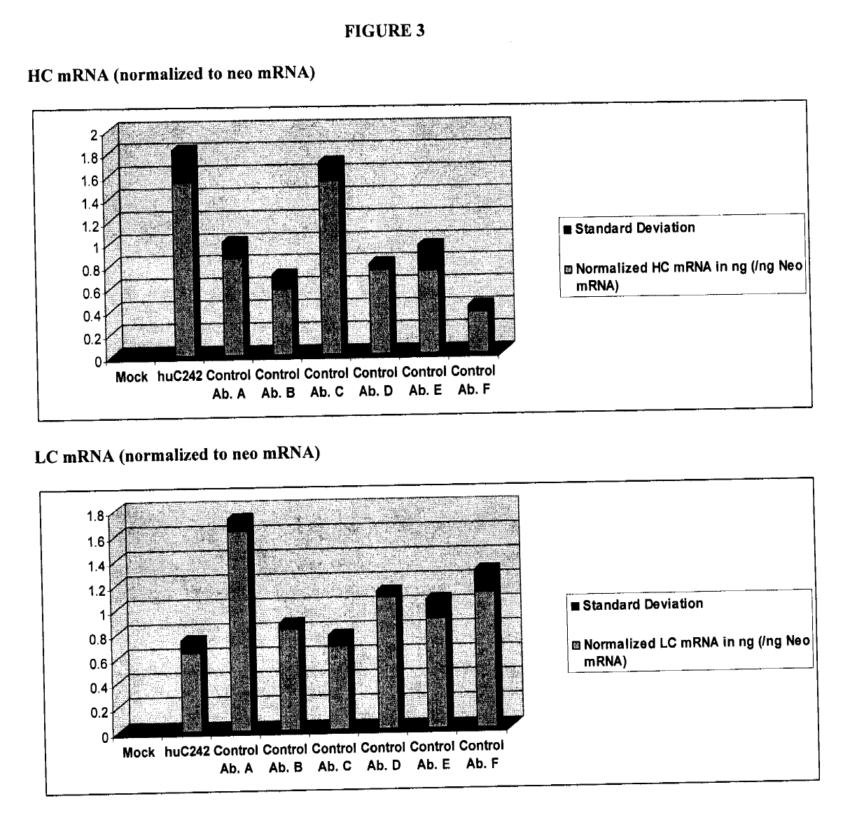

Humanized antibodies are typically produced by having their genes expressed in a mammalian host cell, such as CHO (Chinese hamster ovary) cells or T293 cells (a human kidney cell line). We observed that different humanized antibodies prepared by the resurfacing technology were produced in different amounts in the same mammalian host cells (FIG. 2), although similar amounts of mRNA for the antibodies were produced (FIG. 3). We concluded that the primary amino acid sequence of the variable regions affected the production of the antibodies. Thus we developed a method of improving the productivity in mammalian host cells of humanized monoclonal antibodies that have a core of buried murine amino acids in the variable domain region frameworks.

### ABBREVIATIONS AND DEFINITIONS

MAb monoclonal antibody
CH Constant region (domain) of heavy chain
CH1, CH2, CH3 constant regions 1, 2, & 3 of heavy chain
CL constant region of light chain
VH variable region (domain) of heavy chain
VL variable region (domain) of light chain
CDR Complementarity determining region
CDRL1, CDRL2, CDRL3 complementarity determining regions 1, 2, & 3 of light chain
CDRH1, CDRH2, CDRH3 complementarity determining regions 1, 2, & 3 of heavy chain
FR Framework region (domain) of variable domain
FRL1, FRL2, FRL3. FRL4 Framework regions 1, 2, 3, & 4 of light chain variable domain
FRH1, FRH2, FRH3. FRH4 Framework regions 1, 2, 3, & 4 of heavy chain variable domain
qPCR Quantitative polymerase chain reaction

### Description of Antibodies and Definitions

As shown in FIG. 1, antibodies typically comprise two heavy chains linked together by disulphide bonds and two light chains. Each light chain is linked to a respective heavy chain by a disulphide bond. Each heavy chain comprises in order, starting at the N-terminus, a variable domain (region), a constant domain (region) 1, a hinge region, and constant domains (regions) 2 and 3. Each light chain has a variable domain (region) at the N-terminus and a constant domain at the C-terminus. The light chain variable domain is aligned with the variable domain of the heavy chain. The light chain constant domain is aligned with constant domain 1 of the heavy chain. The constant domains in the light and heavy chains are not involved directly in antigen binding.

The variable domains of each pair of light and heavy chains form the antigen binding site. The domains on the light and heavy chains have the same general structure and each domain comprises a framework of four regions, whose sequences are relatively conserved, connected by three complementarity determining regions (CDRs). The four framework regions of each the LC and HC largely adopt a beta-sheet conformation and the CDRs form loops connecting, and in some cases forming part of, the beta-sheet structure. The six CDRs of a variable region of an antibody (three each from the LC and HC) are held in close proximity to each other and the framework regions and form the antigen binding site. CDRs and framework regions of antibodies may be determined by reference to Kabat (“Sequences of proteins of immunological interest” US Dept. of Health and Human Services, US Government Printing Office, 1987).

Amino acids from the variable regions of the mature heavy and light chains of immunoglobulins are designated Hx and Lx respectively, where x is a number designating the position of an amino acid according to the scheme of Kabat (supra). Kabat lists many amino acid sequences for antibodies for each subgroup (e.g., murine, human, rat etc.). Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the Ln (n being any integer, say e.g., 5) position of a human antibody occupies the equivalent position to an amino acid position L5 of a mouse antibody. Any two antibody sequences can only be aligned in one way, by using the numbering scheme in Kabat (supra). Therefore, for antibodies, percent identity has a unique and well-defined meaning.

As used herein, the term “framework region” refers to those portions of immunoglobulin light and heavy chain variable regions that are relatively conserved (i.e., other than the CDRs) among different immunoglobulins in a genus comprising one or more species, as defined by Kabat, et al., supra.

As used herein, “variant antibody” or a “variant” refers to an antibody that has an amino acid sequence that differs from the amino acid sequence of a parent antibody. Such variants necessarily have less than 100% sequence identity or similarity with the parent antibody. In a preferred embodiment, the variant will have an amino acid sequence that has from about 75% to less than 100% amino acid sequence identity or similarity with the amino acid sequence of either the heavy or light chain variable domain of the parent antibody, more preferably from about 80% to less than 100%, more preferably from about 85% to less than 100%, more preferably from about 90% to less than 100%, and most preferably from about 95% to less than 100%. Identity or similarity with respect to this sequence is defined herein as the percentage of amino acid residues in the candidate sequence that are identical (i.e. same residue) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity. The antibody variant is generally one that has an amino acid substitution in the variable region (by one or more amino acid residues; e.g. by at least one to about twenty-five amino acid residues and preferably by about one to about ten amino acid residues) as compared to the corresponding variable region of a parent antibody.

The “parent” antibody as used herein encompasses an antibody produced by a gene that predominates in a natural population. It also includes an antibody that is of natural mutant form. Further included are antibodies that have been produced or are likely to be produced from such natural population of antibodies or from their natural mutants. Such antibodies include but are not limited to humanized or resurfaced, fully human, or chimerized antibodies or any antibody, which could be created or manipulated pursuant to the teachings of the present invention. Such antibodies generally possess the binding specificity or possess antigen binding residues of the original antibody, but in some instances such antibodies could also have a different binding specificity. For example, the antibody may show an improved binding specificity to an antigen, which is partially related or unrelated to the original antigen.

A non-limiting example of a parent antibody is “parent C242 antibody” which refers to an antibody that has antigen binding residues of, or derived from, the murine C242 antibody (U.S. Pat. No. 5,552,293) or a derivative thereof. For example, the monoclonal antibody C242 may be a murine monoclonal antibody or a humanized, chimerized, fully human, C242 possessing antigen binding residues of murine monoclonal antibody C242.

Target-directed therapy, such as antibody-directed therapy, offers advantages over non-targeted therapy such as systemic therapy via oral or intravenous administration of drugs or whole body therapy such as external radiation therapy (XRT). An advantage of antibody-directed therapy, and of therapy using monoclonal antibodies (MAbs) in particular, is the ability to deliver doses of a therapeutic agent to a tumor, with greater sparing of normal tissue from the effects of the therapeutic agent. This directed therapy uses naked MAbs or MAbs conjugated to cell binding agents, such as drugs, bacterial or other toxins, radionuclides, and neutron-capturing agents, such as boron addends.

The term “epitope” includes any protein determinant capable of specific binding to an immunoglobulin. Epitopic determinants usually consist of chemically active surface groupings of molecules such as amino acids or sugar side chains and usually have specific three dimensional structural characteristics, as well as specific charge characteristics.

An amino acid residue is called a rare variable region framework residue at a given position in the sequence of the framework region, if it is found at this position in less than 10% of all antibody sequences in a large data base of murine antibodies.

An example of a large data base is the Kabat Antibody data base (see e.g. Johnson G, Wu T T. Kabat Database and its applications: future directions. Nucleic Acids Res. 2001 Jan. 1; 29(1):205-6.). A large antibody database contains at least 1000 individual antibody variable region sequences.

With the definitions set forth above, without being bound by a particular embodiment, the following discussion is offered to facilitate understanding of the invention.

The present invention, in one non-limiting aspect, provides a method to enhance the production in mammalian cells of humanized antibodies that have a murine variable region core structure. The method identifies non-consensus amino acid residues in the murine core of the variable region framework and replaces them with the amino acid residue of a murine consensus sequence.

Thus in one embodiment, the present invention encompasses manufacturing of antibody variants or fragments thereof, wherein the variants are manufactured by substituting one or more amino acid residues in a parent antibody with the corresponding residue from a consensus variable region framework sequence. As a consequence of such substitution(s), variant antibodies or fragments thereof show enhanced antibody synthesis when introduced in a host cell as compared to the parent antibody.

In a parent antibody, substitution is preferably made of one or more non-consensus amino acids, identified by aligning the sequence each of the heavy chain and light chain variable domain framework region of a parent antibody with a consensus sequence of the heavy chain and light chain variable domain framework region, with the corresponding consensus sequence amino acid residue.

In a preferred embodiment, the substitution of amino acid residues in a parent antibody is performed in the heavy chain. In another preferred embodiment such amino acid substitution is performed in the light chain. Such substitution in the heavy or the light chain of a parent antibody can be performed independently or simultaneously. The consensus sequence is derived from the sequences of a subgroup of antibodies belonging to the same species (e.g., murine) or across the species from the same genus (e.g., mus or rattus) or from across the genus or other taxonomic classification according to their presumed natural relationships as that to which the antibody from which the parent was derived.

In another embodiment, the invention provides a method for increasing production of a variant antibody or a fragment thereof as compared to a parent antibody in a host cell, the method comprising: a) aligning the sequence each of the heavy chain and light chain variable domain framework region of a parent antibody with a consensus sequence of the heavy chain and light chain variable domain framework region, wherein the consensus heavy or light chain sequence is derived from a database of murine antibody variable domains; b) substituting one or more heavy chain residues in the parent antibody variable domain framework region with a murine heavy chain consensus residue or substituting one or more light chain residues in the parent antibody variable domain framework region with a murine consensus light chain residue wherein the substitution produces the variant antibody or a fragment thereof which when introduced into the host cell is produced at a higher yield as compared to the parent antibody; c) identifying in the parent antibody one or more amino acid residues selected from Q45 or A70 in the light chain, or one or more amino acid residue selected from E16, D26, K46 or T89 in the heavy chain, the amino acid residue determined by the Kabat antibody residue numbering scheme; and d) substituting the one or more amino acid residues in the parent antibody with one or more amino acid residue selected from K45 (optionally, K may be replaced with a non-consensus residue R for the biophysical considerations) or D70, respectively, in the light chain, or one or more amino acid residues selected from A16, G26, E46 or S46 or V89, respectively, in the heavy chain wherein the substitution produces the variant antibody or a fragment thereof which when introduced into the host cell is produced at a higher yield as compared to the parent antibody.

Substitution in the heavy or light chain can be performed independently or simultaneously.

In a preferred embodiment, in the variant antibody light chain Q45 is substituted by K45 (optionally, K may be replaced with a non-consensus residue R for the biophysical considerations) and A70 is substituted by D70 and in the variant antibody heavy chain E16 is substituted by A16; D26 is substituted by G26; K46 is substituted by E46 or S46; and T89 is substituted by V89. Such substitution preferably increases the variant antibody yield by at least about 100% or about 200%. In a preferred embodiment, the yield is at least about 300% or greater. In a more preferred embodiment, the yield is about 400% or greater. In a most favored embodiment, the yield is about 500% or greater. The increase in yield of variant protein may also depend on other factors, such as but not limited to the use of growth factors or the use of serum free medium to culture cells.

The invention also encompasses an isolated nucleic acid comprising a full length murine or human, humanized or chimerized C242 coding sequence having at least one variation in amino acid codons in a region of the sequence encoding for a heavy chain variable region or a light chain variable region, wherein the at least one variation increases the yield of a protein encoded by the C242 gene and wherein the protein includes at least one amino acid variation encoded by the at least one codon variation

In the light chain substitution is selected from framework positions (Kabat numbering scheme):

Q45 to K45; Optionally, K may be replaced with a non-consensus residue R for the biophysical considerations.

A70 to D70

In the heavy chain substitution is selected from framework positions (Kabat numbering scheme):

E16 to A16

D26 to G26

K46 to E46

T89 to V89

Such sequence substitution encodes for a variant C242 gene product, which is a variant antibody.

The invention also encompasses a method for increasing the yield of an antibody, which is a variant of a parent antibody, from a host cell culture by substituting in the parent antibody one or more amino acid residues of SEQ ID NO:1 (heavy chain) or SEQ ID NO:2 (light chain), the method comprising a) aligning SEQ ID NO:1 (heavy chain) with a consensus heavy chain sequence or SEQ ID NO:2 (light chain) with a consensus light chain sequence, wherein the consensus heavy or light chain sequence is derived from a database of murine antibody sequences (e.g. Kabat database—Johnson and Wu, 2001) by aligning heavy and light chain immunoglobulin variable region frameworks using amino acid sequence analysis software such as Vector NTI (Invitrogen); b) b) identifying in the parent antibody one or more amino acid residues selected from, E16, D26, K46 or T89 in the SEQ ID NO.: 1 and Q45 or A70 in the SEQ ID NO:2 the amino acid residue determined by Kabat scheme, and c) substituting one or more of the amino acid residues; E16, D26, K46 or T89 in the SEQ ID NO:1 and Q45 or A70 in the SEQ ID NO:2 with an amino acid residue selected from A16, G26, E46, S46 or V89, respectively, in the SEQ ID NO:1 and K45 (optionally, K may be replaced with a non-consensus residue R for the biophysical considerations) or D70, respectively, in the SEQ ID NO.:2, from the consensus sequence wherein the substitution results in production of a variant antibody and wherein when the variant antibody is introduced into a host cell, the yield of the variant is greater than the parent antibody.

In another embodiment, the invention encompasses a variant antibody or epitope binding fragment thereof, such as a variant of huC242, wherein the variant has one or more amino acid substitutions in a parent antibody having a variable region comprising a heavy chain of SEQ ID NO: 1 [huC242 heavy chain] and a light chain of SEQ ID NO:2 [huC242 light chain] and the variant shows an improved synthesis of the heavy and/or light chain, and improved heavy/light chain assembly when introduced in a single host cell as compared to the parent antibody, wherein the substitution is performed at one or more heavy chain variable region positions selected from 16, 26, 46, or 89 in the SEQ ID NO:1 or light chain variable region positions 45 or 70, in the SEQ ID NO:2, or both, the positions being determined by Kabat numbering scheme. More preferably the variant antibody has amino acid substitution selected from the group consisting of light chain residues Q45 to K45 (optionally, K may be replaced with a non-consensus residue R for the biophysical considerations) or A70 to D70 or heavy chain residues E16 to A16, D26 to G26, K46 to E46, or T89 to V89 and is located in the framework region of the heavy or the light chain.

In another embodiment, the cell binding agent of the present invention also specifically recognizes a ligand, such as the C242 antigen (CD44/CanAg), so that the conjugates will be in contact with the target cell for a sufficient period of time to allow the cytotoxic agent portion of the conjugate to act on the cell, and/or to allow the conjugates sufficient time in which to be internalized by the cell.

In a preferred embodiment, the cytotoxic conjugates comprise a variant of an anti-C242 antibody as the cell binding agent, more preferably the cytotoxic conjugate comprises a variant selected from A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G antibody or an epitope-binding fragment thereof. These antibodies are able to specifically recognize the C242 antigen (CD44/CanAg), and direct the cytotoxic agent to an abnormal cell or a tissue, such as cancer cells, in a targeted fashion.

The second component of the cytotoxic conjugates of the present invention is a cytotoxic agent. The term “cytotoxic agent” as used herein refers to a substance that reduces or blocks the function, or growth, of cells and/or causes destruction of cells.

In preferred embodiments, the cytotoxic agent is a taxol, a maytansinoid such as DM1 or DM4, CC-1065 or a CC-1065 analog. In preferred embodiments, the cell binding agents of the present invention are covalently attached, directly or via a cleavable or non-cleavable linker, to the cytotoxic agent.

In another embodiment, the humanized antibody or an epitope-binding fragment thereof can be conjugated to a drug, such as a maytansinoid, to form a prodrug having specific cytotoxicity towards antigen-expressing cells by targeting the drug to a ligand, such as the C242 antigen (CD44/CanAg). Cytotoxic conjugates comprising such antibodies and a small, highly toxic drug (e.g., maytansinoids, taxanes, and CC-1065 analogs) can be used as a therapeutic for treatment of tumors, such as breast and ovarian tumors

Thus, in one embodiment the antibody variant of the parent antibody produced in accordance to the teachings of the present invention may be used for targeted therapy as naked antibodies or as antibodies acting as cell binding agents.

### Cytotoxic Agents.

The cytotoxic agent used in the cytotoxic conjugate of the present invention may be any compound that results in the death of a cell, or induces cell death, or in some manner decreases cell viability. Preferred cytotoxic agents include, for example, maytansinoids and maytansinoid analogs, taxoids, CC-1065 and CC-1065 analogs, dolastatin and dolastatin analogs, defined below. These cytotoxic agents are conjugated to the antibodies, antibodies fragments, functional equivalents, improved antibodies and their analogs as disclosed herein.

The cytotoxic conjugates may be prepared by in vitro methods. In order to link a drug or prodrug to the antibody, a linking group is used. Suitable linking groups are well known in the art and include disulfide groups, thioether groups, acid labile groups, photolabile groups, peptidase labile groups and esterase labile groups. Preferred linking groups are disulfide groups and thioether groups. For example, conjugates can be constructed using a disulfide exchange reaction or by forming a thioether bond between the antibody and the drug or prodrug.

Maytansinoids

Among the cytotoxic agents that may be used in the present invention to form a cytotoxic conjugate are maytansinoids and maytansinoid analogs. Examples of suitable maytansinoids include maytansinol and maytansinol analogs. Maytansinoids are drugs that inhibit microtubule formation and that are highly toxic to mammalian cells.

Examples of suitable maytansinol analogues include those having a modified aromatic ring and those having modifications at other positions. Examples of some suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Specific examples of suitable analogues of maytansinol having a modified aromatic ring include:

(1) C-19-dechloro (U.S. Pat. No. 4,256,746) (prepared by LAH reduction of ansamytocin P2);

(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and

(3) C-20-demethoxy, C-20-acyloxy (—OCOR), +/−dechloro (U.S. Pat. No. 4,294,757) (prepared by acylation using acyl chlorides).

Specific examples of suitable analogues of maytansinol having modifications of other positions include:

C-9-SH (U.S. Pat. No. 4,424,219) (prepared by the reaction of maytansinol with H₂S or P₂S₅);

C-14-alkoxymethyl (demethoxy/CH₂OR) (U.S. Pat. No. 4,331,598);

C-14-hydroxymethyl or acyloxymethyl (CH2OH or CH2OAc) (U.S. Pat. No. 4,450,254) (prepared from Nocardia);

C-15-hydroxy/acyloxy (U.S. Pat. No. 4,364,866) (prepared by the conversion of maytansinol by Streptomyces);

C-15-methoxy (U.S. Pat. Nos. 4,313,946 and 4,315,929) (isolated from Trewia nudiflora);

C-18-N-demethyl (U.S. Pat. Nos. 4,362,663 and 4,322,348) (prepared by the demethylation of maytansinol by Streptomyces); and

4,5-deoxy (U.S. Pat. No. 4,371,533) (prepared by the titanium trichloride/LAH reduction of maytansinol).

In a preferred embodiment, the cytotoxic conjugates of the present invention utilize the thiol-containing maytansinoid DM1, formally termed N^{2′}-deacetyl-N^{2′}-(3-mercapto-1-oxopropyl)-maytansine, as the cytotoxic agent. DM1 is represented by the following structural formula (I):L (I).

In another preferred embodiment, the cytotoxic conjugates of the present invention utilize the thiol-containing maytansinoid DM4, formally termed N^{2′}-deacetyl-N-^{2′}(4-methyl-4-mercapto-1-oxopentyl)-maytansine as the cytotoxic agent. DM4 is represented by the following structural formula (II):

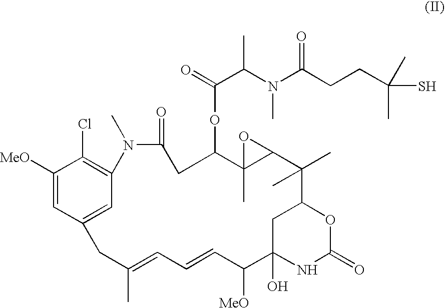

In further embodiments of the invention, other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical, and further wherein one of the substituents can be H, and wherein the acyl group has a linear chain length of at least three carbon atoms between the carbonyl functionality and the sulfur atom.

Such additional maytansines include compounds represented by formula (III):

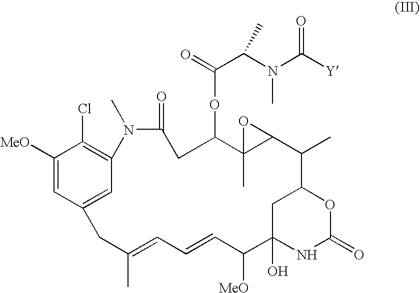

wherein:
Y′ represents

(CR₇R₈)ₗ(CR₉═CR₁₀)ₚ(C≡C)_{q}Aₒ(CR₅R₆)ₘDᵤ(CR₁₁═CR₁₂)ᵣ(C≡C)ₛBₜ(CR₃R₄)ₙC R₁R₂SZ,

wherein:
R₁ and R₂ are each independently linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
A, B, D are cycloalkyl or cycloalkenyl having 3-10 carbon atoms, simple or substituted aryl or heterocyclic aromatic or heterocycloalkyl radical;
R₃, R₄, R₅, R₆, R₇, R₈, R₉, R₁₀, R₁₁, and R₁₂ are each independently H, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
l, m, n, o, p, q, r, s, t and u are each independently 0 or an integer of from 1 to 5, provided that at least two of l, m, n, o, p, q, r, s, t and u are not zero at any one time; and
Z is H, SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (III) include compounds of formula (III) wherein:

R₁ is methyl, R₂ is H and Z is H.

R₁ and R₂ are methyl and Z is H.

R₁ is methyl, R₂ is H, and Z is —SCH₃.

R₁ and R₂ are methyl, and Z is —SCH₃.

Such additional maytansines also include compounds represented by formula (IV-L), (IV-D), or (IV-D,L):

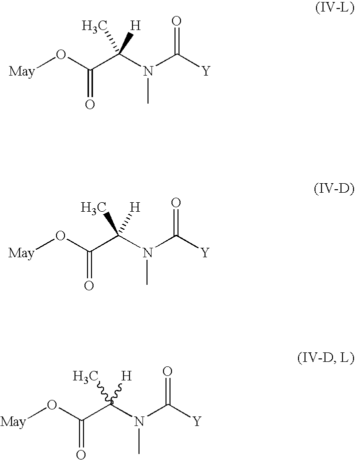

wherein:

Y₁ represents (CR₇R₈)ₗ(CR₅R₆)ₘ(CR₃R₄)ₙCR₁R₂SZ,

wherein:
R₁ and R₂ are each independently linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical, and in addition R₂ can be H;
R₃, R₄, R₅, R₆, R₇ and R₈ are each independently H, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical;
l, m and n are each independently an integer of from 1 to 5, and in addition n can be 0;
Z is H, SR or —COR wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical; and
May represents a maytansinoid which bears the side chain at C-3, C-14 hydroxymethyl, C-15 hydroxy or C-20 desmethyl.

Preferred embodiments of formulas (IV-L), (IV-D) and (IV-D,L) include compounds of formulas (IV-L), (IV-D) and (IV-D,L) wherein:

R₁ is methyl, R₂ is H, R₅, R₆, R₇, and R₈ are each H, l and m are each 1, n is 0,

### and Z is H.

R₁ and R₂ are methyl, R₅, R₆, R₇, R₈ are each H, l and m are 1, n is 0, and Z is H.
R₁ is H, R₂ is methyl, R₅, R₆, R₇, and R₈ are each H, l and m are each 1, n is 0, and Z is —SCH₃.
R₁ and R₂ are methyl, R₅, R₆, R₇, R₈ are each H, l and m are 1, n is 0, and Z is —SCH₃.

Preferably the cytotoxic agent is represented by formula (IV-L).

Such additional maytansines also include compounds represented by formula (V):

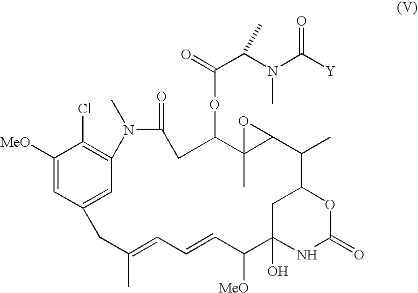

wherein
Y represents (CR₇R₈)ₗ(CR₅R₆)ₘ(CR₃R₄)ₙCR₁R₂SZ,
wherein:
R₁ and R₂ are each independently linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
R₃, R₄, R₅, R₆, R₇ and R₈ are each independently H, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical;
l, m and n are each independently an integer of from 1 to 5, and in addition n can be 0; and
Z is H, SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (V) include compounds of formula (V) wherein:

R₁ is methyl, R₂ is H, R₅, R₆, R₇, and R₈ are each H; l and m are each 1; n is 0; and Z is H.
R₁ and R₂ are methyl; R₅, R₆, R₇, R₈ are each H, l and m are 1; n is 0; and Z is H.
R₁ is methyl, R₂ is H, R₅, R₆, R₇, and R₈ are each H, l and m are each 1, n is 0, and Z is —SCH₃.
R₁ and R₂ are methyl, R₅, R₆, R₇, R₈ are each H, l and m are 1, n is 0, and Z is —SCH₃.

Such additional maytansines further include compounds represented by formula (VI-L), (VI-D), or (VI-D,L)

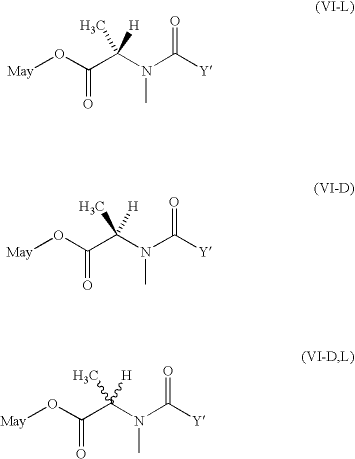

wherein:
Y′ represents

(CR₇R₈)ₗ(CR₉═CR₁₀)ₚ(C≡C)_{q}Aₒ(CR₅R₆)ₘDᵤ(CR₁═CR₁₂)ᵣ(C≡C)ₛBₜ(CR₃R₄)ₙC R₁R₂SZ,

wherein:
R₁ and R₂ are each independently linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
A, B, D are cycloalkyl or cycloalkenyl having 3-10 carbon atoms, simple or substituted aryl or heterocyclic aromatic or heterocycloalkyl radical;
R₃, R₄, R₅, R₆, R₇, R₈, R₉, R₁₀, R₁₁, and R₁₂ are each independently H, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
l, m, n, o, p, q, r, s, t and u are each independently 0 or an integer of from 1 to 5, provided that at least two of l, m, n, o, p, q, r, s, t and u are not zero at any one time;
Z is H, SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical; and
May is a maytansinoid.

Preferred embodiments of formula (VI) include compounds of formula (VI) wherein:

R₁ is methyl, R₂ is H and Z is H.
R₁ and R₂ are methyl and Z is H.
R₁ is methyl, R₂ is H, and Z is —SCH₃.
R₁ and R₂ are methyl, and Z is —SCH₃.

The above-mentioned maytansinoids can be conjugated to variant anti-C242 antibody A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K (R)/K46E; A70D/K46E/D26G; Q45K(R)/D26G; Q45K(R)/K46E/D26G or a homologue or fragment thereof, wherein the antibody is linked to the maytansinoid using the thiol or disulfide functionality that is present on the acyl group of an acylated amino acid side chain found at C-3, C-14 hydroxymethyl, C-15 hydroxy or C-20 desmethyl of the maytansinoid, and wherein the acyl group of the acylated amino acid side chain has its thiol or disulfide functionality located at a carbon atom that has one or two substituents, said substituents being linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition one of the substituents can be H, and wherein the acyl group has a linear chain length of at least three carbon atoms between the carbonyl functionality and the sulfur atom.

A preferred conjugate of the present invention is the one that comprises the variant A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G or a homologue or fragment thereof, conjugated to a maytansinoid of formula (VIII):

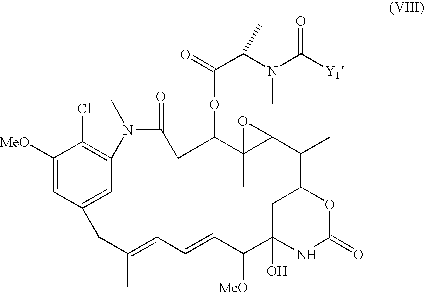

wherein:

Y₁′ represents

(CR₇R₈)ₗ(CR₉═CR₁₀)ₚ(C≡C)_{q}Aₒ(CR₅R₆)ₘDᵤ(CR₁₁═CR₁₂)ᵣ(C≡C)ₛBₜ(CR₃R₄)ₙC R₁R₂S—,

wherein:
R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and in addition R₂ can be H;
A, B, and D, each independently is cycloalkyl or cycloalkenyl having 3-10 carbon atoms, simple or substituted aryl, or heterocyclic aromatic or heterocycloalkyl radical;
R₃, R₄, R₅, R₆, R₇, R₈, R₉, R₁₀, R₁₁, and R₁₂ are each independently H, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical; and
l, m, n, o, p, q, r, s, t and u are each independently 0 or an integer of from 1 to 5, provided that at least two of l, m, n, o, p, q, r, s, t and u are not zero at any one time.

Preferably, R₁ is methyl, R₂ is H or R₁ and R₂ are methyl.

An even more preferred conjugate of the present invention is the one that comprises the variant anti-C242 antibody A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G or a homologue or fragment thereof, conjugated to a maytansinoid of formula (IX-L), (IX-D), or (IX-D,L)

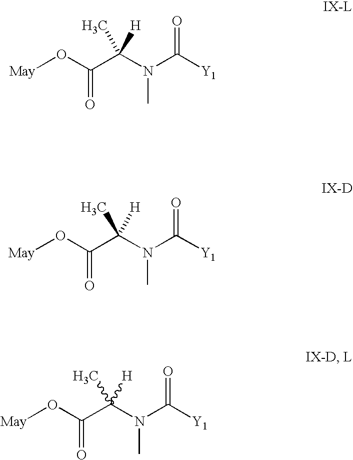

wherein
Y₁ represents (CR₇R₈)ₗ(CR₅R₆)ₘ(CR₃R₄)ₙCR₁R₂S—,
wherein:
R₁ and R₂ are each independently linear alkyl or alkenyl having from 1 to 10 carbon atoms, branched or cyclic alkyl or alkenyl having from 3 to 10 carbon atoms, phenyl, substituted phenyl, heterocyclic aromatic or heterocycloalkyl radical, and in addition R₂ can be H;
R₃, R₄, R₅, R₆, R₇ and R₈ are each independently H, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical;
l, m and n are each independently an integer of from 1 to 5, and in addition n can be 0; and
May represents a maytansinol which bears the side chain at C-3, C-14 hydroxymethyl, C-15 hydroxy or C-20 desmethyl.

Preferred embodiments of formulas (IX-L), (IX-D) and (IX-D,L) include compounds of formulas (IX-L), (IX-D) and (IX-D,L) wherein:

R₁ is methyl, R₂ is H or R₁ and R₂ are methyl,
R₁ is methyl, R₂ is H, R₅, R₆, R₇ and R₈ are each H; l and m are each 1; n is 0,
R₁ and R₂ are methyl; R₅, R₆, R₇ and R₈ are each H; l and m are 1; n is 0.

Preferably the cytotoxic agent is represented by formula (IX-L).

An further preferred conjugate of the present invention is the one that comprises the variant anti-C242 antibody A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G or a homologue or fragment thereof, conjugated to a maytansinoid of formula (X):

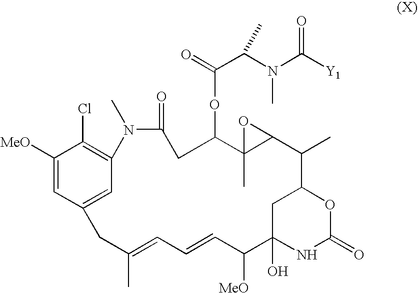

wherein the substituents are as defined for formula (IX) above.

A further preferred conjugate of the present invention is the one that comprises the variant anti-C242 antibody A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G or a homologue or fragment thereof, conjugated to a maytansinoid of formula (XI):

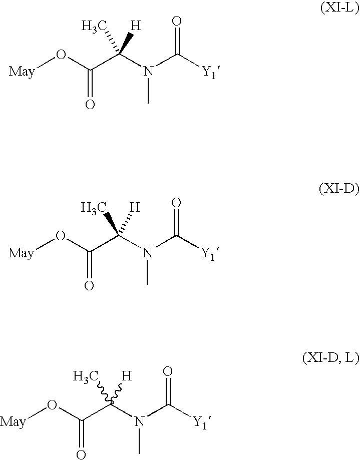

wherein the substituents are as defined for formula (VIII) above.

Especially preferred are any of the above-described compounds, wherein R₁ is H, R₂ is methyl, R₅, R₆, R₇ and R₈ are each H, l and m are each 1, and n is 0.

Further especially preferred are any of the above-described compounds, wherein R₁ and R₂ are methyl, R₅, R₆, R₇, R₈ are each H, l and m are 1, and n is 0

Further, the L-aminoacyl stereoisomer is preferred.

Examples of linear alkyls or alkenyls having from 1 to 10 carbon atoms include, but are not limited to, methyl, ethyl, propyl, butyl, pentyl, hexyl, propenyl, butenyl and hexenyl.

Examples of branched alkyls or alkenyls having from 3 to 10 carbon atoms include, but are not limited to, isopropyl, isobutyl, sec.-butyl, tert.-butyl, isopentyl, 1-ethyl-propyl, isobutenyl and isopentenyl.

Examples of cyclic alkyls or alkenyls having from 3 to 10 carbon atoms include, but are not limited to, cyclopropyl, cyclobutyl, cyclopentyl, cyclohexyl, cyclopentenyl, and cyclohexenyl.

Simple aryls include aryls having 6 to 10 carbon atoms, and substituted aryls include aryls having 6 to 10 carbon atoms bearing at least one alkyl substituent containing from 1 to 4 carbon atoms, or alkoxy substituent such as methoxy, ethoxy, or a halogen substituent or a nitro substituent.

Examples of simple aryl that contain 6 to 10 carbon atoms include phenyl and naphthyl.

Examples of substituted aryl include nitrophenyl, dinitrophenyl.

Heterocyclic aromatic radicals include groups that have a 3 to 10-membered ring containing one or two heteroatoms selected from N, O or S.

Heterocycloalkyl radicals include cyclic compounds, comprising 3 to 10-membered ring systems, containing one or two heteroatoms, selected from N, O, or S.

Examples of heterocyclic aromatic radicals include pyridyl, nitro-pyridyl, pyrollyl, oxazolyl, thienyl, thiazolyl, and furyl.

Examples of heteroalkyl radicals include dihydrofuryl, tetrahydrofuryl, tetrahydropyrollyl, piperidinyl, piperazinyl, and morpholino.

Each of the maytansinoids taught in U.S. Pat. No. 7,276,497 may also be used in the cytotoxic conjugate of the present invention. The entire disclosure of U.S. Pat. No. 7,276,497 is incorporated herein by reference.

Disulfide-Containing Linking Groups

In order to link the maytansinoid to an antibody, such as the variant anti-C242 antibody A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G, the maytansinoid comprises a linking moiety. The linking moiety contains a chemical bond that allows for the release of fully active maytansinoids at a particular site. Suitable chemical bonds are well known in the art and include disulfide bonds, acid labile bonds, photolabile bonds, peptidase labile bonds and esterase labile bonds. Preferred are disulfide bonds.

The linking moiety also comprises a reactive chemical group. In a preferred embodiment, the reactive chemical group can be covalently bound to the maytansinoid via a disulfide bond linking moiety.

Particularly preferred reactive chemical groups are N-succinimidyl esters and N-sulfosuccinimidyl esters.

Particularly preferred maytansinoids comprising a linking moiety that contains a reactive chemical group are C-3 esters of maytansinol and its analogs where the linking moiety contains a disulfide bond and the chemical reactive group comprises a N-succinimidyl or N-sulfosuccinimidyl ester.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. However the C-3 position is preferred and the C-3 position of maytansinol is especially preferred.

While the synthesis of esters of maytansinol having a linking moiety is described in terms of disulfide bond-containing linking moieties, one of skill in the art will understand that linking moieties with other chemical bonds (as described above) can also be used with the present invention, as can other maytansinoids. Specific examples of other chemical bonds include acid labile bonds, photolabile bonds, peptidase labile bonds and esterase labile bonds. The disclosure of U.S. Pat. No. 5,208,020, incorporated herein, teaches the production of maytansinoids bearing such bonds.

The synthesis of maytansinoids and maytansinoid derivatives having a disulfide moiety that bears a reactive group is described in U.S. Pat. Nos. 6,441,163 and 6,333,410, and U.S. Patent Publication No. 2003-0055226 A1, each of which is herein incorporated by reference.

The reactive group-containing maytansinoids, such as DM1, are reacted with a variant anti-C242 antibody A70D; Q45K/R; D26G; K46E; K46E/T89V; K46E/K82S; K46E/E16A/D26G; A70D/K46E/T89V; K46E/D26G; K46E/K82S/D26G; K46E/T89V/D26G; A70D/K46E; Q45K(R)/K46E/T89V; A70D/D26G; Q45K(R)/K46E; A70D/K46E/D26G; Q45K(R)/D26G; Q45K(R)/K46E/D26G, to produce cytotoxic conjugates. These conjugates may be purified by HPLC or by gel-filtration.

Several excellent schemes for producing such antibody-maytansinoid conjugates are provided in U.S. Pat. Nos. 6,333,410, 6,411,163 and 6,716,821 and U.S. Patent Publication No. 2003-0055226 A1, each of which is incorporated herein in its entirety.

In general, a solution of an antibody in aqueous buffer may be incubated with a molar excess of maytansinoids having a disulfide moiety that bears a reactive group. The reaction mixture can be quenched by addition of excess amine (such as ethanolamine, taurine, etc.). The maytansinoid-antibody conjugate can then be purified by gel-filtration.

The number of maytansinoid molecules bound per antibody molecule can be determined by measuring spectrophotometrically the ratio of the absorbance at 252 nm and 280 nm. An average of 1-10 maytansinoid molecules/antibody molecule is preferred.

Conjugates of antibodies with maytansinoid drugs can be evaluated for their ability to suppress proliferation of various unwanted cell lines in vitro. For example, cell lines such as the human epidermoid carcinoma line A-431, the human small cell lung cancer cell line SW2, the human breast tumor line SKBR3 and the Burkitt's lymphoma line Namalwa can easily be used for the assessment of cytotoxicity of these compounds. Cells to be evaluated can be exposed to the compounds for 24 hours and the surviving fractions of cells measured in direct assays by known methods. IC₅₀ values can then be calculated from the results of the assays.

PEG-Containing Linking Groups

Maytansinoids may also be linked to antibodies using PEG linking groups, as set forth in U.S. Pat. No. 6,716,821. These PEG linking groups are soluble both in water and in non-aqueous solvents, and can be used to join one or more cytotoxic agents to a cell binding agent. Exemplary PEG linking groups include hetero-bifunctional PEG linkers that bind to cytotoxic agents and cell binding agents at opposite ends of the linkers through a functional sulfhydryl or disulfide group at one end, and an active ester at the other end.

As a general example of the synthesis of a cytotoxic conjugate using a PEG linking group, reference is again made to U.S. Pat. No. 6,716,821 for specific details. Synthesis begins with the reaction of one or more cytotoxic agents bearing a reactive PEG moiety with an antibody, resulting in displacement of the terminal active ester of each reactive PEG moiety by an amino acid residue of the antibody, to yield a cytotoxic conjugate comprising one or more cytotoxic agents covalently bonded to an antibody through a PEG linking group.

Other Linking Groups

Maytansinoid conjugates comprising non-cleavable linkers are described in U.S. Patent Publication No. 2005-0169933 A1, the entire disclosure of which is incorporated herein by reference.

Taxanes

The toxic agent used in the cytotoxic conjugates according to the present invention may also be a taxane or derivative thereof.

Taxanes are a family of compounds that includes paclitaxel (Taxol), a cytotoxic natural product, and docetaxel (Taxotere), a semi-synthetic derivative, two compounds that are widely used in the treatment of cancer. Taxanes are mitotic spindle poisons that inhibit the depolymerization of tubulin, resulting in cell death. While docetaxel and paclitaxel are useful agents in the treatment of cancer, their antitumor activity is limited because of their non-specific toxicity towards normal cells. Further, compounds like paclitaxel and docetaxel themselves are not sufficiently potent to be used in conjugates of cell binding agents.

A preferred taxane for use in the preparation of cytotoxic conjugates is the taxane of formula (XI):

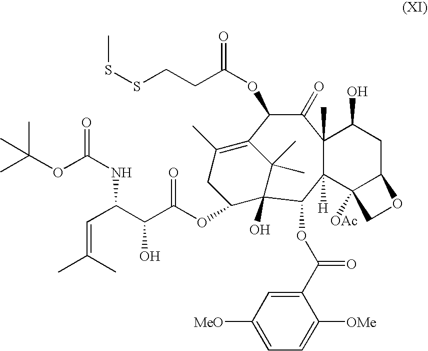

Methods for synthesizing exemplary taxanes that may be used in the cytotoxic conjugates of the present invention, along with methods for conjugating the taxanes to cell binding agents such as antibodies, are described in detail in U.S. Pat. Nos. 5,416,064, 5,475,092, 6,340,701, 6,372,738, 6,436,931, 6,596,757, 6,706,708 and 6,716,821 and in U.S. Patent Publication No. 2004-0024049 A1.

CC-1065 Analogues

The toxic agent used in the cytotoxic conjugates according to the present invention may also be CC-1065 or a derivative thereof.

CC-1065 is a potent anti-tumor antibiotic isolated from the culture broth of Streptomyces zelensis. CC-1065 is about 1000-fold more potent in vitro than are commonly used anti-cancer drugs, such as doxorubicin, methotrexate and vincristine (B. K. Bhuyan et al., Cancer Res., 42, 3532-3537 (1982)). Non-limiting examples of suitable CC-1065 and its analogs for use in the present invention are disclosed in U.S. Pat. Nos. 6,372,738, 6,340,701, 5,846,545 and 5,585,499.

The cytotoxic potency of CC-1065 has been correlated with its alkylating activity and its DNA-binding or DNA-intercalating activity. These two activities reside in separate parts of the molecule. Thus, the alkylating activity is contained in the cyclopropapyrroloindole (CPI) subunit and the DNA-binding activity resides in the two pyrroloindole subunits.

Although CC-1065 has certain attractive features as a cytotoxic agent, it has limitations in therapeutic use. Administration of CC-1065 to mice caused a delayed hepatotoxicity leading to mortality on day 50 after a single intravenous dose of 12.5 μg/kg {V. L. Reynolds et al., J. Antibiotics, XXIX, 319-334 (1986)}. This has spurred efforts to develop analogs that do not cause delayed toxicity, and the synthesis of simpler analogs modeled on CC-1065 has been described {M. A. Warpehoski et al., J. Med. Chem., 31, 590-603 (1988)}.

In another series of analogs useful in the present invention, the CPI moiety was replaced by a cyclopropabenzindole (CBI) moiety {D. L. Boger et al., J. Org. Chem., 55, 5823-5833, (1990), D. L. Boger et al., BioOrg. Med. Chem. Lett., 1, 115-120 (1991)}. These compounds maintain the high in vitro potency of the parental drug, without causing delayed toxicity in mice. Like CC-1065, these compounds are alkylating agents that bind to the minor groove of DNA in a covalent manner to cause cell death. However, clinical evaluation of the most promising analogs, Adozelesin and Carzelesin, has led to disappointing results {B. F. Foster et al., Investigational New Drugs, 13, 321-326 (1996); I. Wolff et al., Clin. Cancer Res., 2, 1717-1723 (1996)}. These drugs display poor therapeutic effects because of their high systemic toxicity.

The therapeutic efficacy of CC-1065 analogs can be greatly improved by changing the in vivo distribution through targeted delivery to the tumor site, resulting in lower toxicity to non-targeted tissues, and thus, lower systemic toxicity. In order to achieve this goal, conjugates of analogs and derivatives of CC-1065 with cell-binding agents that specifically target tumor cells have been described {U.S. Pat. Nos. 5,475,092; 5,585,499; 5,846,545}. These conjugates typically display high target-specific cytotoxicity in vitro, and exceptional anti-tumor activity in human tumor xenograft models in mice {R. V. J. Chari et al., Cancer Res., 55, 4079-4084 (1995)}.

Methods for synthesizing CC-1065 analogs that may be used in the cytotoxic conjugates of the present invention, along with methods for conjugating the analogs to cell binding agents such as antibodies, are described in detail in U.S. Pat. Nos. 5,475,092, 5,846,545, 5,585,499, 6,534,660, 6,586,618 and 6,756,397 and in U.S. Patent Publication No. 2003-0195365 A1.

Other Drugs

Drugs such as methotrexate, daunorubicin, doxorubicin, vincristine, vinblastine, melphalan, mitomycin C, chlorambucil, calicheamicin, tubulysin and tubulysin analogs, duocarmycin and duocarmycin analogs, dolastatin and dolastatin analogs, such as auristatins and analogs, are also suitable for the preparation of conjugates of the present invention. The drug molecules can also be linked to the antibody molecules through an intermediary carrier molecule such as serum albumin. Doxarubicin and Danorubicin compounds, as described, for example, in U.S. Ser. No. 09/740,991, may also be useful cytotoxic agents. These drugs could also be used for co-therapy as discussed below.

### Co-Therapy

The phrase “combination therapy” (or “co-therapy”), means the use of a variant antibody, such as a variant of huC242 antibody, a chemotherapeutic agent, or an immunotoxin, and is intended to embrace administration of each agent in a sequential manner in a regimen that will provide beneficial effects of the drug combination. Cotherapy is intended as well to embrace co-administration of these agents in a substantially simultaneous manner, such as by ingestion of a single dosage having a fixed ratio of these active agents or ingestion of multiple, separate medicaments for each agent. “Combination therapy” also includes simultaneous or sequential administration by intravenous, intramuscular or other parenteral routes into the body, including direct absorption through mucous membrane tissues, as found in the sinus passages. Sequential administration also includes drug combinations where the individual elements may be administered at different times and/or by different routes but which act in combination to provide a beneficial effect.

The phrase “therapeutically-effective” is intended to qualify the amount of each agent for use in the combination therapy which will achieve the goal of improvement in reducing or preventing tumor, for example, the progression of tumors, while avoiding adverse side effects typically associated with each agent.

A preferred combination therapy would consist essentially of two or more active agents, namely, a variant huC242 naked antibody or a conjugate thereof and other agent selected from an immunotoxin, a chemotherapeutic agent, an immunomodulator or an antibody, which differs from the variant antibody. The agents would be used in combination in a weight ratio range from about 0.5-to-one to about twenty-to-one of the naked antibody or the conjugate thereof to any one of the other agents. A preferred range of these two agents would be from about one-to-one to about fifteen-to-one, while a more preferred range would be from about one-to-one to about five-to-one, depending ultimately on the selection of the antibody or conjugate and any one of the other agent. Depending on the needs of the patient, the ratio for the antibody or the conjugate thereof, and the other agent could also be reversed. For example, after the initial treatment, if a patient is found to be more responsive to a higher dose of one of the other agents as compared to the variant naked antibody or a conjugate thereof, a provider of such medication can switch the ratio to make the treatment more effective. The preparation of immunotoxins is generally well known in the art (see, e.g., U.S. Pat. No. 4,340,535, incorporated herein by reference). Each of the following patents and patent applications are further incorporated herein by reference for the purposes of even further supplementing the present teachings regarding immunotoxin generation, purification and use: U.S. Pat. Nos. 5,855,866; 5,776,427; 5,863,538; 6,004,554; 5,965,132; 6,051,230; and 5,660,827; and U.S. application Ser. No. 07/846,349.

Variant antibodies, such as a huC242 variant, can also be bound directly or indirectly to an immunomodulator. Among the biological response modifiers (such as an immunomodulator) that in some way can increase the destruction of the tumor by the antibody of this invention are included lymphokines such as: Tumor Necrosis Factor, Macrophage Activator Factor, Colony Stimulating Factor, Interferons, etc.

When the cotherapy involves an injectable complex, it may further comprise a therapeutic agent selected from the group consisting of hormones, immunosuppressants, antibiotics, cytostatics, diruretics, gastro-intestinal agents, cardiovascular agents, anti-inflammatory agents, analgesics, local anesthetics, and a neuropharmacological agent, wherein these agents are administered to lower the risk of any side effect.

### Therapeutic Composition

The invention also relates to a therapeutic composition for the treatment of a hyperproliferative disorder in a mammal which comprises a therapeutically effective amount of a variant antibody of the invention and a pharmaceutically acceptable carrier. In one embodiment the pharmaceutical composition is for the treatment of cancer of the lung, breast, colon, prostate, kidney, pancreas, ovary, cervix and lymphatic organs, osteosarcoma, synovial carcinoma, a sarcoma or a carcinoma in which CanAg is expressed, and other cancers yet to be determined in which CanAg is expressed predominantly. In another embodiment, the pharmaceutical composition relates to the treatment of other disorders such as, for example, autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as mV infection, HIV infection, AIDS, etc.; and parasite infections, such as giardiasis, amoebiasis, schistosomiasis, and others as determined by one of ordinary skill in the art.

The instant invention provides pharmaceutical compositions comprising:

an effective amount of a variant antibody or epitope binding fragment thereof or a conjugate of a cytotoxic agent and a variant antibody or epitope binding fragment thereof of the present invention, and

a pharmaceutically acceptable carrier, which may be inert or physiologically active.

As used herein, “pharmaceutically-acceptable carriers” include any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combinations thereof. In many cases, it will be preferable to include isotonic agents, such as sugars, polyalcohols, or sodium chloride in the composition. In particular, relevant examples of suitable carrier include: (1) Dulbecco's phosphate buffered saline, pH˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The compositions herein may also contain a further therapeutic agent, as necessary for the particular disorder being treated. Preferably, the variant antibody or epitope binding fragment thereof or a conjugate of a cytotoxic agent and the variant antibody or epitope binding fragment thereof of the present invention, and the supplementary active compound will have complementary activities, that do not adversely affect each other. In a preferred embodiment, the further therapeutic agent is an antagonist of fibroblast-growth factor (FGF), hepatocyte growth factor (HGF), tissue factor (TF), protein C, protein S, platelet-derived growth factor (PDGF), or HER2 receptor.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the variant antibody or epitope binding fragment thereof or a conjugate of a cytotoxic agent and the variant antibody or epitope binding fragment thereof of the present invention in the required amount in the appropriate solvent, followed by sterilization by microfiltration. As solvent or vehicle, there may be used water, saline, phosphate buffered saline, dextrose, glycerol, ethanol, and the like, as well as combinations thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The variant antibody or epitope binding fragment thereof or a conjugate of a cytotoxic agent and the variant antibody or epitope binding fragment thereof of the present invention may also be orally administered. As solid compositions for oral administration, tablets, pills, powders (gelatine capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used; they are generally between 5 mg and 1000 mg per day orally for an adult with unit doses ranging from 1 mg to 250 mg of active substance. In general, the doctor will determine the appropriate dosage depending on the age, weight and any other factors specific to the subject to be treated.

The improved or the variant antibodies or the conjugates thereof of the present invention can be used in therapeutic formulations as an agonist or antagonist that are prepared for storage by mixing the variants or the conjugates thereof having the desired degree of purity with optional physiologically acceptable carriers, excipients or stabilizers [Remington's Pharmaceutical Sciences 16th edition, Osol, A. Ed. (1980); U.S. patent application Ser. No. 10/846,129], in the form of aqueous solutions or lyophilized formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g., Zn-protein complexes); and/or non-ionic surfactants such as TWEEN™, PLURONICS™ or polyethylene glycol (PEG).

The variants may also be formulated in liposomes. Liposomes containing the molecule of interest are prepared by methods known in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556.

Particularly useful immunoliposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al., J. Biol. Chem. 257:286-288 (1982) via a disulfide interchange reaction. A chemotherapeutic agent (such as Doxorubicin) is optionally contained within the liposome. See Gabizon et al., J. National Cancer Inst. 81(19):1484 (1989).

The formulation herein may also contain more than one active compound as necessary for the particular indication being treated, preferably those with complementary activities that do not adversely affect each other. Such molecules are suitably present in combination in amounts that are effective for the purpose intended. For example, a C242 variant or a conjugate thereof may be combined with a co-therapeutic agent, such as, chemotherapeutic agent.

The active ingredients may also be entrapped in microcapsules prepared, for example, by coacervation techniques or by interfacial polymerization, for example, hydroxymethylcellulose or gelatin-microcapsule and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes, as discussed above.

Sustained-release preparations may be prepared. Suitable examples of sustained-release preparations include semipermeable matrices of solid hydrophobic polymers containing the antagonist, which matrices are in the form of shaped articles, e.g., films, or microcapsules. Examples of sustained-release matrices include polyesters, hydrogels [for example, poly(2-hydroxyethyl-methacrylate), or poly(vinylalcohol)], polylactides (U.S. Pat. No. 3,773,919), copolymers of L-glutamic acid and ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### Therapeutic Methods of Use

In another embodiment, the present invention provides a method for inhibiting the C242 antigen activity by administering an antibody which antagonizes the function of anti-CD44/CanAg (antigen), to a patient in need thereof. Any of the variant antibody or epitope binding fragment thereof or a conjugate of a cytotoxic agent and the variant antibody or epitope binding fragment thereof of the invention, may be used therapeutically.

In a preferred embodiment, the variant antibodies or epitope binding fragment thereof or conjugate of a cytotoxic agent and the variant antibodies or epitope binding fragments thereof of the invention are used for the treatment of a hyperproliferative disorder in a mammal. In a more preferred embodiment, one of the pharmaceutical compositions disclosed above, and which contains a variant antibody or epitope binding fragment thereof or a conjugate of a cytotoxic agent and a variant antibody or epitope binding fragment thereof of the invention, is used for the treatment of a hyperproliferative disorder in a mammal. Preferably, the disorder is a cancer of the lung, breast, colon, prostate, kidney, pancreas, ovary, cervix and lymphatic organs, osteosarcoma, synovial carcinoma, a sarcoma or a carcinoma in which CanAg is expressed, and other cancers yet to be determined in which CanAg is expressed predominantly. In another embodiment, said pharmaceutical composition relates to other disorders such as, for example, autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as mV infection, HIV infection, AIDS, etc.; and parasite infections, such as giardiasis, amoebiasis, schistosomiasis, and others as determined by one of ordinary skill in the art.

Similarly, the present invention provides a method for inhibiting the growth of selected cell populations comprising contacting target cells, or tissue containing target cells that express CanAg, with an effective amount of a variant antibody or epitope binding fragment thereof or a conjugate of a cytotoxic agent and the variant antibody or epitope binding fragment thereof of the present invention, or a therapeutic agent comprising a variant antibody or epitope binding fragment thereof or a conjugate of a cytotoxic agent and a variant antibody or epitope binding fragment thereof, either alone or in combination with other cytotoxic or therapeutic agents.

The method for inhibiting the growth of selected cell populations can be practiced in vitro, in vivo, or ex vivo. As used herein, “inhibiting growth” means slowing the growth of a cell, decreasing cell viability, causing the death of a cell, lysing a cell and inducing cell death, whether over a short or long period of time.

Examples of in vitro uses include treatments of autologous bone marrow prior to their transplant into the same patient in order to kill diseased or malignant cells; treatments of bone marrow prior to its transplantation in order to kill competent T cells and prevent graft-versus-host-disease (GVHD); treatments of cell cultures in order to kill all cells except for desired variants that do not express the target antigen; or to kill variants that express undesired antigen.

The conditions of non-clinical in vitro use are readily determined by one of ordinary skill in the art.

Examples of clinical ex vivo use are to remove tumor cells or lymphoid cells from bone marrow prior to autologous transplantation in cancer treatment or in treatment of autoimmune disease, or to remove T cells and other lymphoid cells from autologous or allogeneic bone marrow or tissue prior to transplant in order to prevent graft versus host disease (GVHD). Treatment can be carried out as follows. Bone marrow is harvested from the patient or other individual and then incubated in medium containing serum to which is added the variant antibody or epitope binding fragment thereof or a conjugate of a cytotoxic agent and the variant antibody or epitope binding fragment thereof of the invention. Concentrations range from about 10 μM to 1 pM, for about 30 minutes to about 48 hours at about 37° C. The exact conditions of concentration and time of incubation, i.e., the dose, are readily determined by one of ordinary skill in the art. After incubation the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

For clinical in vivo use, the variant antibody or epitope binding fragment thereof or a conjugate of a cytotoxic agent and the variant antibody or epitope binding fragment thereof of the invention will be supplied as solutions that are tested for sterility and for endotoxin levels. Examples of suitable protocols of cytotoxic conjugate administration are as follows. Conjugates are given weekly for 4 weeks as an i.v. bolus each week. Bolus doses are given in 50 to 100 ml of normal saline to which 5 to 10 ml of human serum albumin can be added. Dosages will be 10 μg to 1 g per administration, i.v. (range of 100 ng to 10 mg/kg per day). More preferably, dosages will range from 50 μg to 30 mg. Most preferably, dosages will range from 1 mg to 20 mg. After four weeks of treatment, the patient can continue to receive treatment on a weekly basis. Specific clinical protocols with regard to route of administration, excipients, diluents, dosages, times, etc., can be determined by one of ordinary skill in the art as the clinical situation warrants.

### Diagnostic

The antibodies or antibody fragments of the invention can also be used to detect C242 antigen (anti-CD44/CanAg) in a biological sample in vitro or in vivo. In one embodiment, the variant C242 antibodies of the invention are used to determine the level of anti-CD44/CanAg in a tissue or in cells derived from the tissue. In a preferred embodiment, the tissue is a diseased tissue. In a preferred embodiment of the method, the tissue is a tumor or a biopsy thereof. In a preferred embodiment of the method, a tissue or a biopsy thereof is first excised from a patient, and the levels of anti-CD44/CanAg in the tissue or biopsy can then be determined in an immunoassay with the antibodies or antibody fragments of the invention. The tissue or biopsy thereof can be frozen or fixed. The same method can be used to determine other properties of the anti-CD44/CanAg, such as its post-translation modification (e.g., glycolation), cell surface levels, or cellular localization.

The above-described method can be used to diagnose a cancer in a subject known to or suspected to have a cancer, wherein the level of CanAg measured in said patient is compared with that of a normal reference subject or standard. Said method can then be used to determine whether a tumor expresses CanAg, which may suggest that the tumor will respond well to treatment with the antibodies, antibody fragments or antibody conjugates of the present invention. Preferably, the tumor is a cancer of the lung, breast, colon, prostate, kidney, pancreas, ovary, cervix and lymphatic organs, osteosarcoma, synovial carcinoma, a sarcoma or a carcinoma in which CanAg is expressed, and other cancers yet to be determined in which CanAg is expressed predominantly.

The present invention further provides for variant monoclonal antibodies, variant humanized antibodies and epitope-binding fragments thereof that are further labeled for use in research or diagnostic applications. In preferred embodiments, the label is a radiolabel, a fluorophore, a chromophore, an imaging agent or a metal ion.

A method for diagnosis is also provided in which said labeled antibodies or epitope-binding fragments thereof are administered to a subject suspected of having a cancer, and the distribution of the label within the body of the subject is measured or monitored.

### Kit

The present invention also includes kits, e.g., comprising a described cytotoxic conjugate and instructions for the use of the cytotoxic conjugate for killing of particular cell types. The instructions may include directions for using the cytotoxic conjugates in vitro, in vivo or ex vivo.

Typically, the kit will have a compartment containing the cytotoxic conjugate. The cytotoxic conjugate may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the cytotoxic conjugate prior to administering to a patient, and tools that aid in administering the conjugate to a patient.

### Designing of Consensus Sequence

The consensus sequences were created by tallying the residues present for each position in the Kabat murine antibody sequence database. Several thousand light and heavy chain variable region sequences were aligned with the ClustalW module in the sequence analysis and data management software “Vector NTI” (A product of Invitrogen Corp.). See also Lu G, Moriyama E N Vector NTI, a balanced all-in-one sequence analysis suite. Brief Bioinform. 2004 December; 5(4):378-88. The alignment was imported into a Microsoft Excel spreadsheet to calculate which residue was most frequently found at each position in the murine light and heavy chain variable region databases and then output the results as the “consensus” sequences. On occasions, consensus could be split between two amino acid residues, wherein the lesser of the two conserved amino acid is chosen to enhance the biophysical properties or humanization considerations of an antibody. For example, in the instant case, in the light chain Q45 is replaced by R which does not replace the non-consensus residue with the murine consensus residue, which is K. Such observations are apparent to one skilled in the art without undue experimentation or needing these specific details.

The Kabat sequence database is commercially available by purchasing a license. Further, thousands of murine antibody sequences are publicly available for download on the NCBI website and can be used to generate similar data.

All references cited herein and in the examples that follow are expressly incorporated by reference in their entireties.

The broad scope of this invention is best understood with reference to the following examples, which are not intended to limit the invention to specific embodiments.

### EXAMPLES

### Materials

The pSynC242 and control plasmid preps were prepared by standard CsCl₂ purification techniques. QuikChange Site-Directed Mutagenesis System was obtained from Stratagene (#200518). RNeasy Mini Kit was ordered from Qiagen (#74104). Superscript First Strand Synthesis System for reverse transcriptase reactions was from GibcoBRL (#11904-418). Cyber Green real time PCR Master Mix was obtained from Applied Biosystems (#4309155). Flourescencein Conjugated Streptavidin was from Jackson Immuno Research (# 016-010-084 1 mg/ml). 96 well U bottom plates were from FALCON (#3077). EZ-Link Sulfo-NHS-LC-Biotin was from Pierce (#21335).

Methods

Amino acid Substitution on huC242 HC and LC Frameworks by Site-Directed Mutagenesis

### Primer Design

Complementary PCR primer pairs for the mutagenesis reactions were 30 bases in length and contained the desired nucleotide(s) substitution in the middle of the primers. The primers where PAGE purified and reconstituted at 150 ng/μl.

PCR Mutagenesis Reactions

PCR reactions were prepared as follows: 5 μl of 10× reaction buffer (Stratagene), 20 ng of dsDNA template, 0.85 μl (125 ng) of forward primer, 0.85 μl (125 ng) of reverse primer, 1 μl of 400 mM dNTP (Stratagene), and ddH2O was added to a final volume of 50 μl in a thin walled epidorf tube. Finally, 1 μl of PfuTurbo DNA polymerase (2.5 U/μl, Stratagene) was added to the reaction mix and the tubes were placed in an MJ Research thermal cycler. The reactions conditions were as follows:

1 cycle at 95° C. for 30 seconds
12 cycles of 95° C. for 30 seconds, followed by 55° C. for 1 minute, and 68° C. for 11 minutes (1 minute/kb with an 11 kb template). The number of cycles was increased to 14 when two bases were to be changed.
1 cycle at 68° C. for 8 minutes

### Hold at 4° C.

Transformation of Competent Cells was Performed as Follows:

The PCR template DNA was neutralized by digestion with the methylation dependant restriction enzyme, Dpn1 (Stratagene). The restriction digest was performed with 1 μl of Dpn1 added directly to the PCR reaction and incubated at 27° C. for 1 hour. The reaction was then transformed by adding 3 μl of the Dpn1 digested PCR product to 50 μl of XL-1 competent cells (Stratagene), incubating on ice for 30 minutes, followed by a 42° C. heat pulse for 45 seconds, returning to ice for 2 minutes, and then adding 0.5 ml of SOC buffer for a final incubation at 37° C. for 1 hour while shaking at 225 rpm. The transformed cells were plated on LB/Amp plates (300 μl per plate) and incubated at 37° C. overnight.

Confirmation of the Mutagenesis

Plasmid DNA was isolated from the transformed colonies with Qiagen mini prep columns and screened by agarose gel electrophoresis. Plasmids that maintained the same electrophoretic mobility as the template DNA were further analyzed by sequencing the VH and VL regions in order to confirm the expected mutagenesis products. Sequencing reactions were performed by standard automated sequencing techniques (Sterky F, Lundeberg J Sequence analysis of genes and genomes. J. Biotechnol. 2000 Jan. 7; 76(1): 1-31).

Transient Transfections

Transient transfection with the antibody expression plasmids were performed with Qiagen's Superfection reagent kit. To ensure transfection equivalency among the test plasmids, DNA concentrations were measuring by OD₂₆₀ and confirmed by visualization on an agarose gel. Prior to transfection, 3×10⁵ 293T cells were plated per well in 6 well dishes. Transfection mixes were made up to 0.6 ml per well with 2 μg of plasmid DNA (or TE for blank control), mixed with 15 μl of Superfect (Qiagen) and incubated at RT for 10 min. The mix was added to the 293T cells which were then placed in a 37° C., 5% CO2 incubator for 2 hr. Next, the cells were washed with cell culture medium and finally incubated in 1 ml of culture medium for 0 hr, 14 hr, 22 hr and 48 hr in a 37° C., 5% CO₂ incubator to allow for antibody production. Secreted antibodies were collected from the culture medium at 0 hr, 14 hr, 22 hr and 48 hr after transfection. Antibody concentration was measured by anti-huIgG1 quantitative ELISA (QE).

Quantitative ELISA's

Quantitative ELISA's were performed in 96-well plates coated with a sheep-anti human IgG antibody (The Binding Site Limited, UK, product code AU0006) for 1 hour at room temperature. The plates were then blocked with 1% neonatal goat serum in PBS for 1 hour at room temperature. Transfection supernatants were serial diluted and applied to the blocked plates followed by another incubation at room temperature for 1 hr. The ELISA plates were then washed 4 times with PBS./0.05% Teen-20, secondary antibody was added (peroxidase-conjugated anti-human Kappa antibody (The Binding Site Limited, UK, product code AP015), and the plates were again incubated for 1 hour at room temperature. Finally the plates were washed 4 times with PBS./0.05% Teen-20 and ABTS was added (0.5 mg/ml ABTS, 0.03% H2O2 in 0.1 M citrate buffer). The ELISA plate absorbance was read at OD₄₀₅ and the human IgG1 concentration was calculated relative to the absorbance of internal standards.

Quantitative Comparison of mRNA and Intracellular HC/LC Levels of Original and Mutated huC242

Total RNA Isolation

Total RNA was isolated from 6×10⁶ transient transfected 293T cells with Qiagen RNeasy spin columns following the kit protocols. Cells were trypsinized, washed with PBS, pelleted, supernatant was removed and pellets were frozen at −80° C. overnight. Cell pellets were disrupted in 500 μl of Buffer RLT containing 1% (v/v) β-mercaptoethanol, mixed thoroughly, and then homogenized by passing though a 20-gauge needle 5 times. To each homogenate, 500 ul of 70% ethanol was added, the tube was mixed, then 700 μl of sample was applied to an RNeasy mini column, and the tubes were spun at 10,000 rpm for 15 seconds. The flow-through was discarded and the column was washed with 350 μl Buffer RW1 follow by another spin at 10,000 rpm for 15 seconds. Cellular DNA was removed with 80 μl of DNase I solution (10 μl DNase I in 70 μl Buffer RDD) added to the center of the column membrane, incubated at RT (20-30° C.) for 15 min., then 350 μl Buffer RW1 was added to the column and the tubes were spun at 10,000 rpm for 15 seconds. The flow through was discarded and the column was transferred into a new 2 ml collection tube for two additional washes with 500 μl Buffer RPE each and spun at 10,000 rpm for 15 seconds for the first wash and 2 minutes for the second wash. The column was placed in a new 2 ml collection tube and spun at full speed for 1 minute to completely dry the column. Finally, the column was transferred to a new 1.5 ml collection tube and the RNA was eluted with 30 μl RNase free water and spun at 10,000 rpm for 1 minute. The RNA concentration was measured by OD260 and then the samples were stored at −80° C.

cDNA Synthesis

First strand cDNA synthesis reactions were performed with Superscript II reverse transcriptase (Invitrogen), following the kit protocols. The reaction mix was made with 3 μg total RNA, 3 μl random hexamers, 1 μl 10 mM dNTP, and brought up to 10 μl with DEPC treated water. The mix was incubated at 65° C. for 5 minutes, and then put on ice for at least 1 minute. To each reaction, 2 μl 10×RT buffer, 4 μl 25 mM MgCl2, 2 μl 0.1M DT T, and 1 μl RNaseOUT ribonuclease inhibitor was added, mixed, and incubated at 25° C. for 2 min. Next, 1 μl (50 U) Superscript II reverse transcriptase was added to each tube, mixed and incubated at 25° C. for 10 minutes, 42° C. for 50 min, 70° C. for 15 min and then chilled on ice. The reaction was collected by a brief spin and the RNA was removed with RNase H (1 μl added to each tube and incubated at 37° C. for 20 minutes) before proceeding with the quantitative PCR.

Quantitative PCR Set Up

Quantitative PCR reactions were performed in 96 well plates using the Cyber Green Real Time PCR Master Mix Kit (Applied Biosystems). The reverse transcriptase reactions were diluted 1:500 and 10 μl of each sample was plated in triplicate wells. For each primer pair, a standard curve (4-5 points) was generated using 10 μl of 1:100, 1:200, 1:400, 1:800 and 1:1600 dilutions of one RNA sample. Internal transfection controls were also performed in triplicate for each sample with primers specific to the neomysin resistance gene present on each plasmid. A separate cell number control was also performed for each sample using actin specific primers. To each experimental and control well, 15 μl of reaction mix (0.05 μl of each 100 μM primer, 12.5 μl of 2× Cyber Green PCR Master Mix and 2.4 μl of ddH2O) was added. The plates were sealed and spun to collect the contents before being placed in the ABI prism 7000 real time thermal cycler. The reactions were performed at 95° C. for 10 minutes followed by 40 cycles of 95° C. for 15 seconds and 60° C. for 1 minute. The reaction data was analyzed with ABI prism 7000 software.

Analysis of Intracellular Heavy and Light Chain Levels

Cells stably or transiently expressing IgGs were lysed in RIPA buffer and the protein concentrations were normalized. Cell lysates were subjected to electrophoresis under either denaturing or non-denaturing conditions. The IgGs in the lysates were purified or concentrated by Protein A precipitation techniques prior to electrophoresis as needed. The acrylamide gels were analyzed by Western blotting techniques using anti-human IgG and anti-human LCκ antibodies to visualize the heavy chain and light chain bands respectively.

### Measurement of Binding Affinity of HuC242 to Antigen-Positive Cells

Non-Competitive Binding

The huC242 and the variant antibodies were normalized to 1-2 μg/ml with FACS buffer (2.5% NGS in RPMI medium). Duplicate 50 μl samples were applied to a 96-well plate and serial diluted 1:1 in FACS buffer. Colo205 cells were resuspended in FACS buffer to 4×10⁶ cells/ml and 50 μl were added to each well. The plate was incubated for 2 hours and then washed 2 times with 200 μl FACS buffer per well followed by spinning at 4° C. for 5 minutes at 1200 rpm. The supernatants were removed and. 50 μl of FITC-labeled secondary antibody (1:100 diluted in FACS buffer) was added to each well and the plate was incubated at 4° C. for 30 minutes. Finally, the plate was washed as described above and the cells were fixed with 200 μl/well of 1% Formaldehyde in PBS. The relative antibody binding to the target cells was analyzed by fluorescence on a BD FACScalliber flow cytometer.

Competitive Binding

HuC242 was biotinylated with 1 mg/ml of EZ-Link Sulfo-NHS-LC-Biotin (Pierce #21335) incubated at room temperature for 1 hour. The reaction was quenched with taurine and then dialysed against 1×PBS at 4° C. over night. The biotinylated huC242 was diluted to 1.6×10⁻⁹ M for a final reaction concentration at 4×10⁻¹⁰ M.

The unlabeled huC242 and variant antibodies were serial diluted 1:2 from 4×10⁻⁸ M to 1×10⁻¹ M and 50 μl of each sample was added to the plate. Next, 50 μl per well of biotinylated huC242 was mixed in by pipetting up and down 3 times. Colo 205 cells were washed two times with FACS buffer, resuspended at 2×10⁵ cells/ml, and 100 μl was mixed into each well containing the antibody mixtures. The plate was incubated on ice for 2 hours, washed 2 times with FACS buffer and then 50 μl of 1:100 diluted Flourescencein Conjugated Streptavidin was added. Next, the plate was incubated on ice for 1 hour, washed 2 times with FACS buffer and the cells were fixed with 200 μl per well of 1% formaldehyde in PBS. The competitive binding was analyzed on a BD FACScalliber flow cytometer.

Low huC242 Production is Observed Within Hours after Transient Transfection of 293T Cells

Transient transfections were performed in 293T cells to compare the expression of huC242 with control antibodies known to have moderate to high expression potentials. Plasmids encoding the two control humanized antibodies as well as huC242 were transfected into 293T cells in parallel and secreted antibodies were collected from the culture medium at 0 hr, 14 hr, 22 hr and 48 hr after transfection. As early as fourteen hours after transfection, the secreted huC242 was already much lower than both control antibodies (FIG. 2), and the difference increased overtime. At 48 hours the accumulated huC242 yield was only about 7% of Control A and 12% of Control B.

The huC242 Heavy and Light Chain mRNA Levels in 293T Transient Transfections Appear Normal

In order to examine whether the low huC242 production is due to low IgG messenger RNA levels, heavy and light chain mRNAs of huC242 were compared to other humanized antibodies in ImmunoGen's repertoire. HuC242 and the control antibodies were transfected into 293T cells in parallel and mRNAs were isolated from the cells after 72 hr. The samples were analyzed by quantitative RT-PCR techniques and the results (FIG. 3) indicate that huC242 mRNA levels are comparable to the control antibodies. The normalized huC242 heavy chain mRNA was somewhat higher than most of the antibodies, but was similar to control C. The huC242 light chain mRNA was lower than that of control A, but similar to controls A and C. Taken together, the cellular mRNA levels of both the huC242 heavy and light chains were found to be comparable with several other antibodies capable of high productivity.

The Relative Ratio of Assembled huC242 (H2L2) to HC(H) is Significantly Lower than the huB4 Ratio in Stable CHO Cell Lines

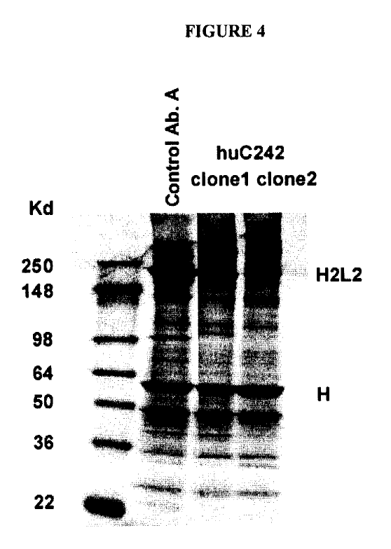

Based on the qPCR results, the low productivity of huC242 is likely to be post-transcriptional. To analyze the post-transcriptional events, intracellular expression and assembly of heavy and light chain peptides were compared between control A and huC242. A stable CHO cell line expressing control A was compared with two stable CHO cell lines expressing huC242. Whole cell lysates were subjected to protein A purification and the isolated IgGs were separated on a non-denaturing gel and stained with Coomassie Blue (FIG. 4). The presence of multiple incomplete assembly species for huC242 compared with the control antibody suggests that inefficient heavy and light chain assembly, perhaps due to poor compatibility between the peptides or insufficient light chain supply, may be a root of low expression of the huC242 antibody.

Multiple huC242 Heavy and Light Chain Framework Residues do not Conform to the Consensus Sequences

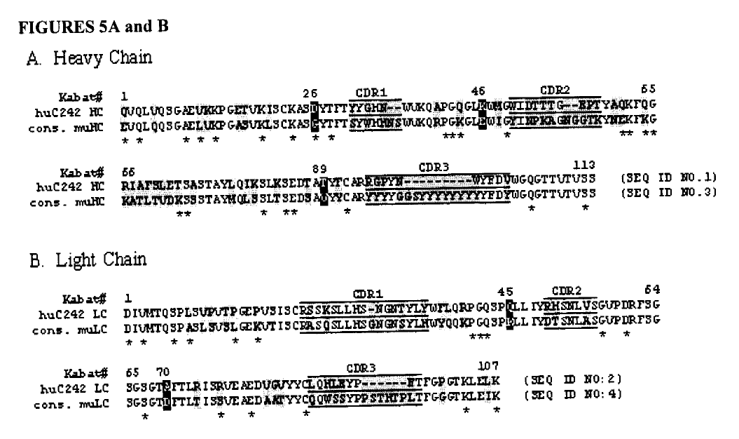

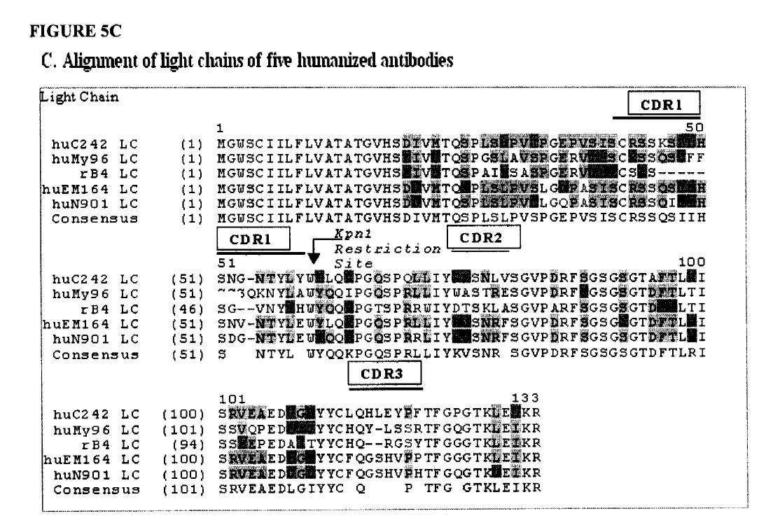

The presence of non-consensus residues in the huC242 heavy and light chain frameworks was initially realized by aligning the huC242 variable region amino acid sequences with control resurfaced antibodies known to be capable of high level expression in stable CHO lines. Since residues that lead to low expression are not likely to be prevalent in nature, the huC242 heavy and light chain variable region frameworks were aligned with consensus sequences generated from the entire Kabat murine IgG1 and Kappa light chain databases respectively (FIG. 5). The murine consensus sequences were utilized because the buried residues in a resurfaced antibody, such as huC242, retain all of the murine residues of the original murine parental antibody and the human surface residues would not generally be considered for substitution. Residue 26 (D), shown in FIG. 5 is an exception to that being a buried residue because it is exposed on the surface. This residue was replaced with G because to begin with it (D) is a murine residue and as a general rule murine residues may be replaced even if they are found outside of the buried residues. The G residue from the murine consensus in this case happens to be consistent with the human sequence used for the C242 humanization.

The same huC242 framework positions that did not match a small set of recombinant antibodies also did not match the consensus sequence from the broad database. Furthermore, many of these huC242 residues were found to be extremely rare in the same positions in the Kabat database, ranging from present in 16% of murine antibodies down to as little as 0.8% (see Table below). These rare, buried framework residues were chosen for further investigation into whether any contributes to the low expression potential of the huC242 antibody.

TABLE
Rare amino acid residues in the variable region framework of huC242
and the frequency of their appearance in the Kabat antibody database.
For comparison, the corresponding amino acid residues of the consensus
sequence and their frequency I given.

| | | | Consensus sequence from | |
|---|---|---|---|---|
| | huC242 | | Kabat | |
| | Amino | Frequency in | Amino | Frequency in |
| Residue No. | acid | Kabat | acid | Kabat |

HEAVY CHAIN VARIABLE REGION FRAMEWORK

| | | | | |
|---|---|---|---|---|
| 16 | E | 95/3408: | A | 1533/3408: |
| | | 2.8% | | 45.6% |
| 26 | D | 29/3661: | G | 3612/2661: |
| | | 0.8% | | 98.7% |
| 46 | K | 88/3715: | E | 3563/3715: |
| | | 2.4% | | 95.9% |
| 82A | K | 17/3745: | S | 1953/3745: |
| | | 0.45% | | 52.1% |
| 89 | T | 648/3894: | V | 2147/3894: |
| | | 16.6% | | 55.1% |

LIGHT CHAIN VARIABLE REGION FRAMEWORK

| | | | | |
|---|---|---|---|---|
| 45 | Q | 244/2606: | K/R | 2307/2606: |
| | | 9.4% | | 88.5% |
| 70 | A | 39/2987: | D | 1652/2987: |
| | | 1.3% | | 55.3% |

Moderate Increase of IgG Production by a Single Amino Substitution in Either the huC242 or Light Chain Frameworks

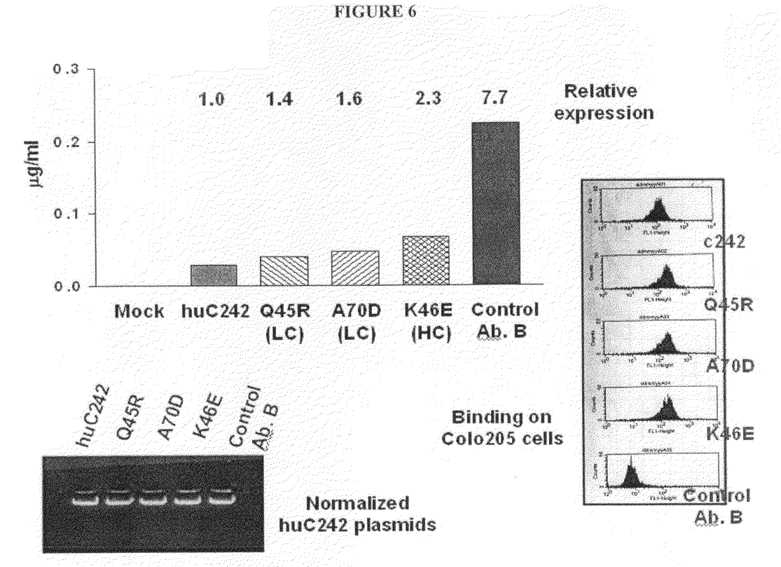

To investigate whether the uncommon residues identified in the huC242 heavy and light chain frameworks may negatively impact the antibody production, these residues were substituted by the corresponding consensus residues using site-directed mutagenesis. The antibody expression for the single amino acid variants of huC242 were compared to huC242 and control antibodies by transient transfection. The respective expression plasmids were transfected into 293T cells and after 72 hours, the levels of secreted IgGs were determined by quantitative ELISA. Moderate increases of IgG production were achieved by single amino acid substitutions in either the huC242 heavy or light chain framework regions (FIG. 6).

To ensure the residue substitutions did not alter antibody binding activity, the huC242 variants were evaluated by FACS on antigen positive Colo 205 cells. The results showed that the amino acid substitutions did not change the binding profile (FIG. 6).

Significant Increases in Antibody Productivity are Achieved by the Combination of Two to Three Residue Changes in the huC242 Heavy and Light Chain Framework

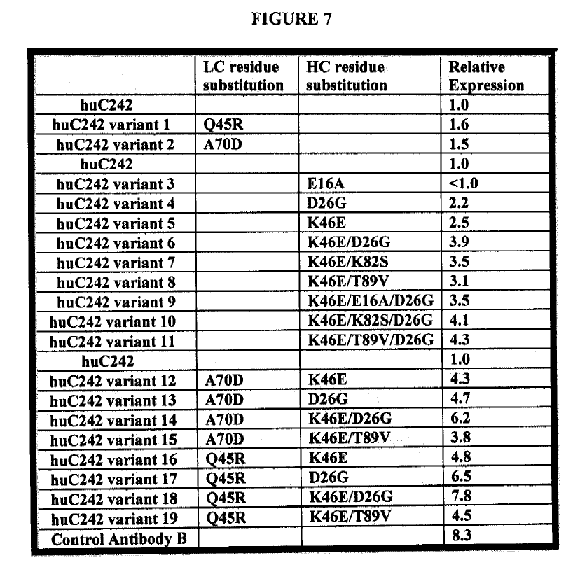

The huC242 amino acid substitutions were expanded to include multiple framework residue changes. The variant heavy and light chain constructs were also mixed and matched to build an array of huC242 variant pairs, each containing two or more residue substitutions. The relative productivities of the huC242 variants were compared in 293T transient transfections as described above. The various residue substitution combinations resulted in different levels of productivity, with the largest increases seen in those containing two or three changes combined between both the huC242 heavy and light chain variable region framework (FIG. 7).

The mRNA levels of the huC242 variant heavy and light chains remain unchanged

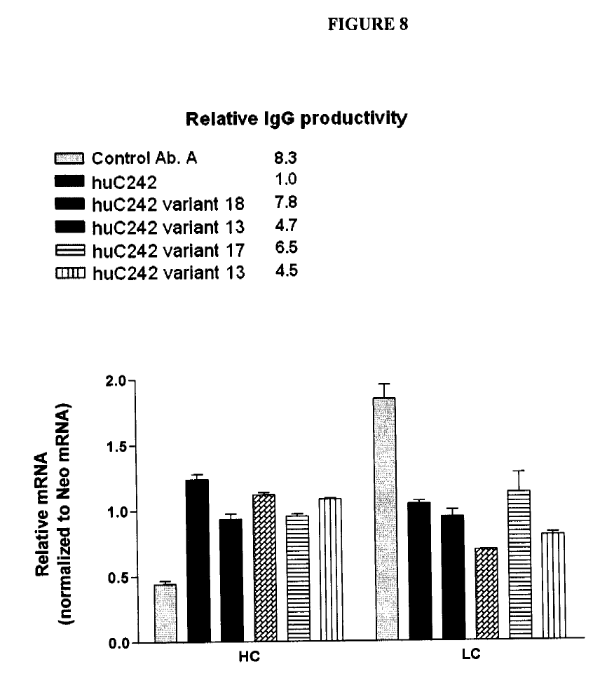

The increase in huC242 productivity seen with the variant sequences could be due to improved transcription, translation or post-translational properties of the antibody. To investigate the source of the increased huC242 expression, a qPCR experiment was conducted evaluating the mRNA levels of huC242 and the huC242.sequence variants expressed in transiently transfected 293T cells. The results indicate that the huC242 variants produced similar levels of mRNA for both heavy and light chain as compared with huC242 (FIG. 8). This suggests that the increased antibody production of the huC242 variants was not due to increased transcription or mRNA stability, but perhaps due to improved post-transcriptional properties.

### An Increase in Intracellular Light Chain Peptide is Observed as a Result of Heavy Chain Variations

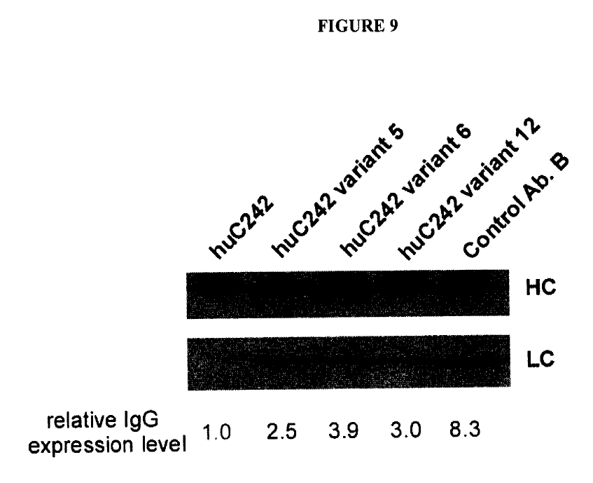

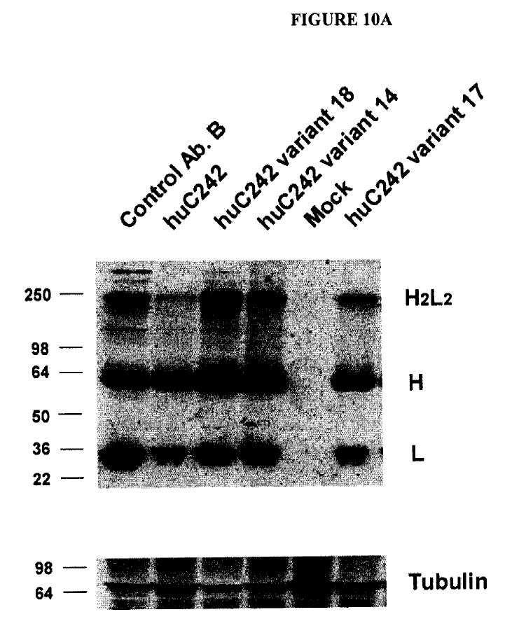

To further study the possible mechanisms associated with the increased huC242 productivity by variable region framework residue substitutions, the intracellular heavy and light chain peptide levels were compared. The huC242 and sequence variants were transiently transfected in 293T cells which were lysed 72 hours later, and whole cell lysates were evaluated by Western blotting techniques under denaturing conditions. Both anti-huIgG1 and anti-huK secondary antibodies were utilized to visualize both the heavy and light chain bands on the same gel. Interestingly, residue substitutions in the heavy chain variable region framework did not affect heavy chain expression but rather increased intracellular accumulation of the huC242 light chain containing no residue substitutions (FIG. 9). These results suggest that the huC242 heavy chain variants protect the huC242 light chain from being degraded, perhaps through enhanced heavy chain light chain compatibility that leads to increased antibody assembly and ultimately increased antibody production. These results also showed that the productivities of the huC242 variants were roughly proportional to their intracellular light chain, but not the heavy chain levels.

The huC242 Residue Substitutions Lead to Improved Heavy and Light Chain Assembly

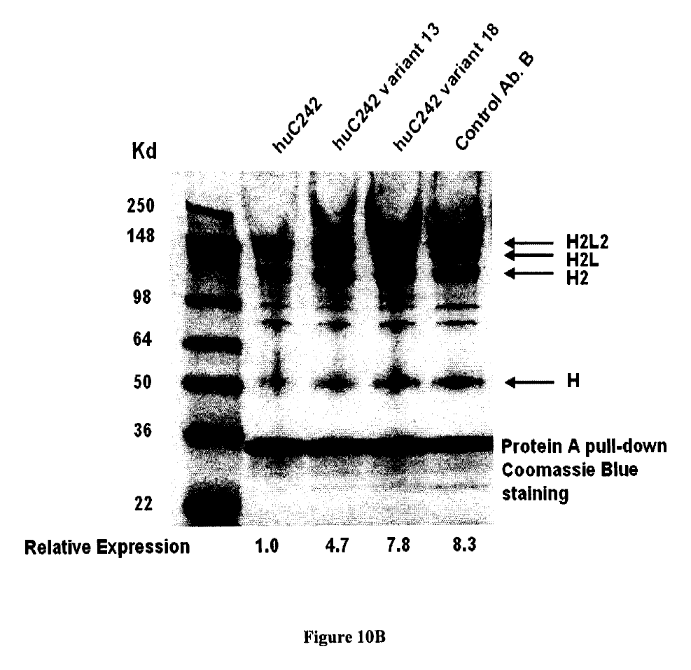

Western blots performed under non-denaturing conditions provide further evidence for improved post-translational properties in the huC242 variants. Whole cell lysates from 293T cells transiently transfected with huC242 and the variant constructs were evaluated by non-denaturing Western blotting techniques (FIG. 10). In these experiments, the non-denatured, fully assembled antibody could be visualized together with the unassembled heavy and light chain as well as intermediate assembly products. The intracellular proportion of fully assemble IgG (H2L2) verses intermediate assembly products (H2, H2L1, etc.) was significantly improved as a result of the amino acid substitutions (FIG. 10(a)). In addition, these results showed that when heavy and light chain variants were used in combination, intracellular levels of both chains were increased. This suggests that the improved interactions between the heavy and light chain variant sequences results in mutual intrachain protection from degradation.

The huC242 and variant constructs were evaluated on Coomassie Blue stained gels to avoid potential artifacts associated with Western blotting techniques (FIG. 10(b)). The whole cell lysates from transiently transfected 293T cells were subjected to Protein A purification techniques, then the isolated IgGs were applied to non-denaturing PAGE and subsequently stained with Coomassie Blue. The results are consistent with those observed by Western blot, demonstrating significant levels of partially assembled antibody in the huC242 lane and an increase in the proportion of fully assembled antibody (H2L2) in the huC242 variant lanes

The Residue Substitutions in the huC242 Variants do not Affect Antigen Binding Activity

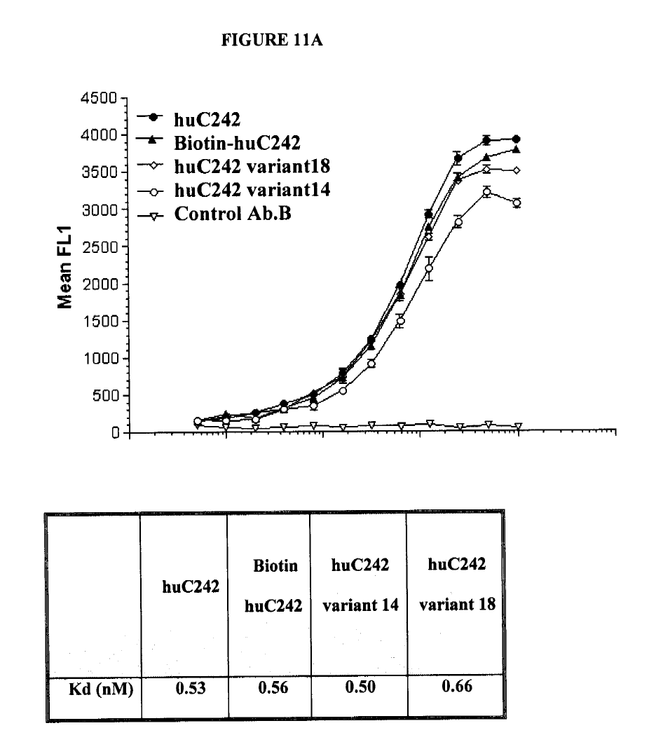

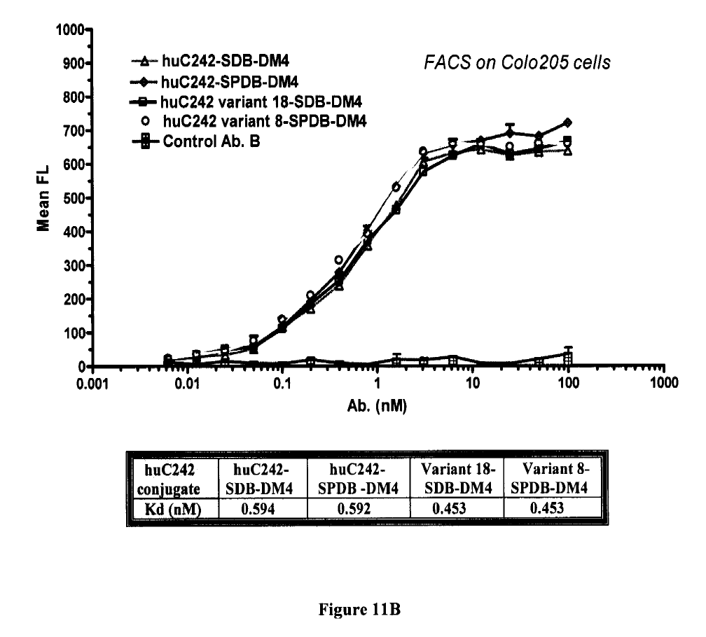

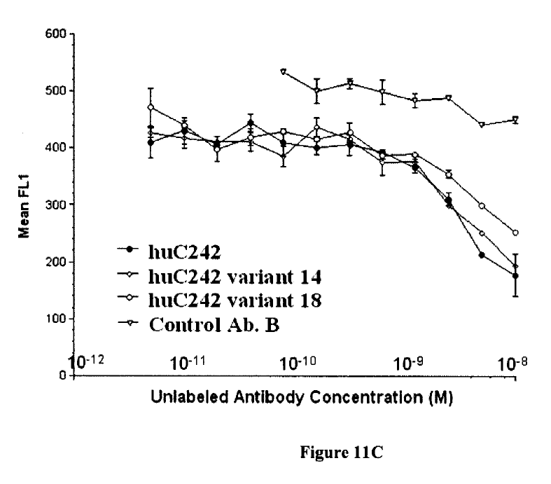

The relative binding activity of huC242 and the variant constructs were evaluated on antigen positive Colo205 cells by FACS. The huC242 variants capable of enhanced antibody productivity did not exhibit significant changes in antigen binding activity (FIG. 11(a)). To further confirm these results, a competitive binding assay was performed by challenging biotinylated huC242 bound to Colo205 cells with serially diluted, unlabeled huC242 variants (FIG. 11(b)). This experiment demonstrated that the huC242 variants could compete with huC242 for binding to antigen positive Colo205 cells in a concentration-dependent manner. The combined results of direct and competitive binding assays indicate that huC242 and the variant constructs have similar binding activities.

Those of ordinary skill in the art will appreciate that the reengineering conditions and techniques, including processes, other than those that are specifically illustrated herein can be employed in the practice of this invention as claimed without resort to undue experimentation. Those of ordinary skill in the art will recognize and understand that functional equivalents of the procedures, processing conditions, and techniques illustrated herein exist in the art. All such known equivalents are intended to be encompassed by this invention.

What is claimed is:

1. A method for increasing production of a humanized murine antibody or a fragment thereof in a host cell by sequence reengineering comprising:
a) aligning a collection of murine antibody variable region framework sequences, wherein such alignment identifies amino acid residues most frequently found (consensus residues) at each position in said framework;
b) comparing said consensus residues with the corresponding residues in the humanized antibody variable region framework sequence;
c) identifying in said humanized antibody one or more non-consensus amino acid residues in the variable region framework sequence; and
d) substituting in said humanized antibody or fragment thereof one or more non-consensus amino acid residues with the consensus residue at the equivalent position to produce a variant antibody, wherein said variant antibody is produced in said host cell at a higher yield as compared to said humanized antibody; and
e) optionally, one or more amino acids is replaced with a non-consensus residue for biophysical considerations.

2. The method of claim 1, wherein said higher yield is at least 2-fold or greater.

3. The method of claim 1, wherein said substitution is in said heavy chain.

4. The method of claim 1, wherein said substitution is in said light chain.

5. The method of claim 1, wherein said substitution is in both the heavy and light chains.

6. The method of any one of claims 3, 4 and 5, wherein the substitution is in the core of the variable region framework sequence.

7. The method of any one of claims 3, 4 and 5, wherein the non-consensus amino acid is a rare amino acid.

8. The method of claim 1, wherein the humanized antibody is huC242 and the substitutions are at one or more heavy chain variable region positions selected from 16, 26, 46, or 89 in the SEQ ID NO:1 or light chain variable region positions 45 or 70 in the SEQ ID NO:2, or both, the positions being determined by Kabat numbering scheme, and wherein the amino acid sequence of the light chain variable region is represented by SEQ ID NO: 2 and the amino acid sequence of the heavy chain variable region is represented by SEQ ID NO:1.

9. The method of claim 1, wherein the humanized antibody is huC242 and the substitutions are one or more selected from the group consisting of Q45K/R and A70D in the light chain, and one or more selected from the group consisting of amino acid residues EIA, D26G, K46E and T89V in the heavy chain, the amino acid residue being determined by the Kabat antibody residue numbering scheme, and wherein the amino acid sequence of the light chain variable region is represented by SEQ ID NO: 2 and the amino acid sequence of the heavy chain variable region is represented by SEQ ID NO:1.

10. The method of claim 1, wherein the humanized antibody is huC242 and the substitutions are one or more selected from the group consisting of Q45K/R and A70D in the light chain, the amino acid residue being determined by the Kabat antibody residue numbering scheme, and wherein the amino acid sequence of the light chain variable region is represented by SEQ ID NO: 2.

11. The method of claim 1, wherein the humanized antibody is huC242 and the substitutions are one selected from the group consisting of amino acid residues E1A, D26G, K46E, and T89V in the heavy chain, the amino acid residue being determined by the Kabat antibody residue numbering scheme, and wherein the amino acid sequence of the heavy chain variable region is represented by SEQ ID NO:1.

12. The method of claim 1, wherein the humanized antibody is huC242 and the substitutions are one selected from the group consisting of amino acid residues E1A, D26G, K46E, T89V, K46E/D26G, K46E/K82S, K46E/T89V, K46E/E16A/D26G, K46E/K82S/D26G and K46E/T89V/D26G in the heavy chain, the amino acid residue being determined by the Kabat antibody residue numbering scheme, and wherein the amino acid sequence of the heavy chain variable region is represented by SEQ ID NO:1.

13. The method of claim 1, wherein the humanized antibody is huC242 and the substitutions are one of A70D or Q45K/R in the light chain and one of K46E, D26G, K46E/D26G or K46E/T89V in the heavy chain, the amino acid residue being determined by the Kabat antibody residue numbering scheme, and wherein the amino acid sequence of the light chain variable region is represented by SEQ ID NO: 2 and the amino acid sequence of the heavy chain variable region is represented by SEQ ID NO:1.

14. The method of any one of claims 8 to 13, wherein said higher yield is at least 2-fold or greater.

15. An antibody produced by the method of any one of claims 1 and 8-13.

16. An isolated nucleic acid comprising a full length human C242 coding sequence having at least one variation in a region of said sequence encoding a heavy chain variable region or a light chain variable region, wherein said at least one variation increases the yield of a protein encoded by said C242 gene and wherein said protein includes said at least one variation.

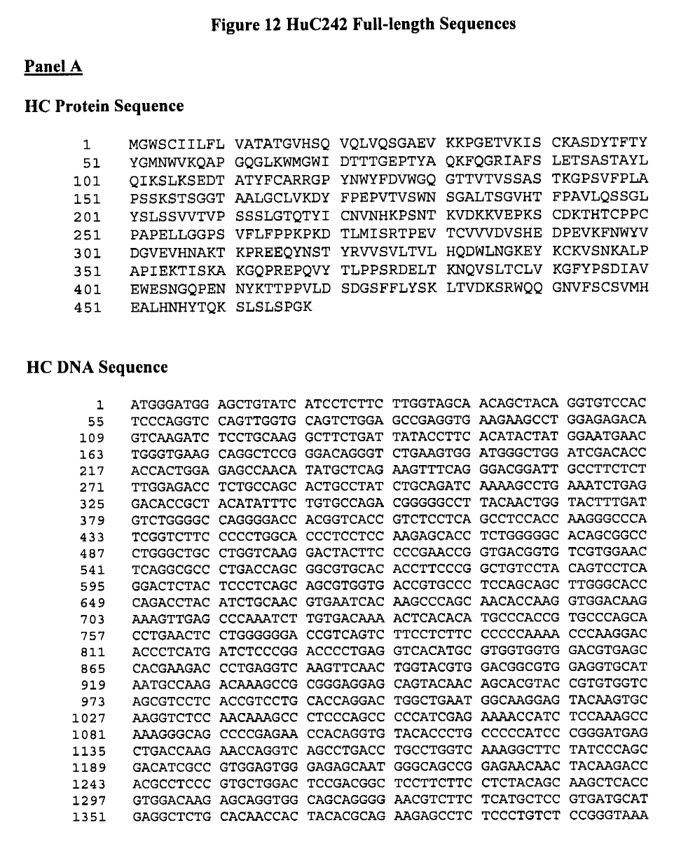

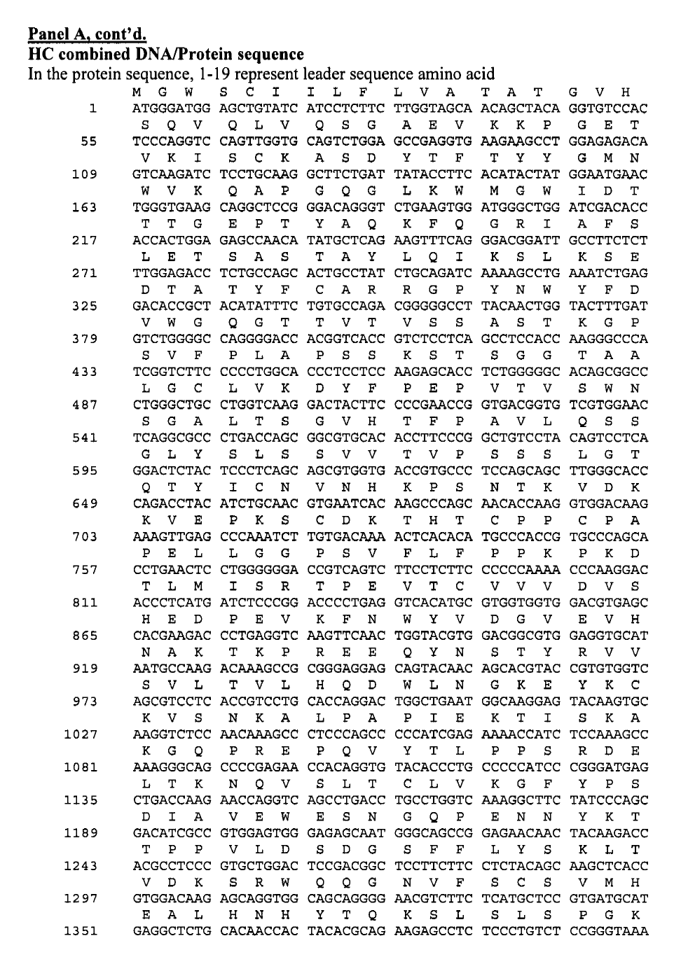

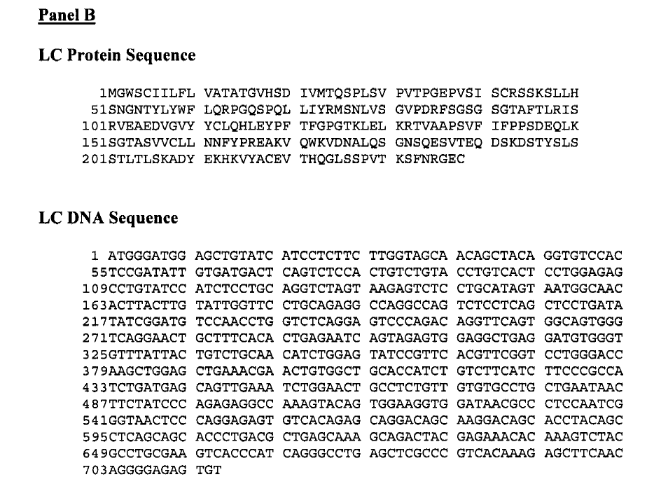

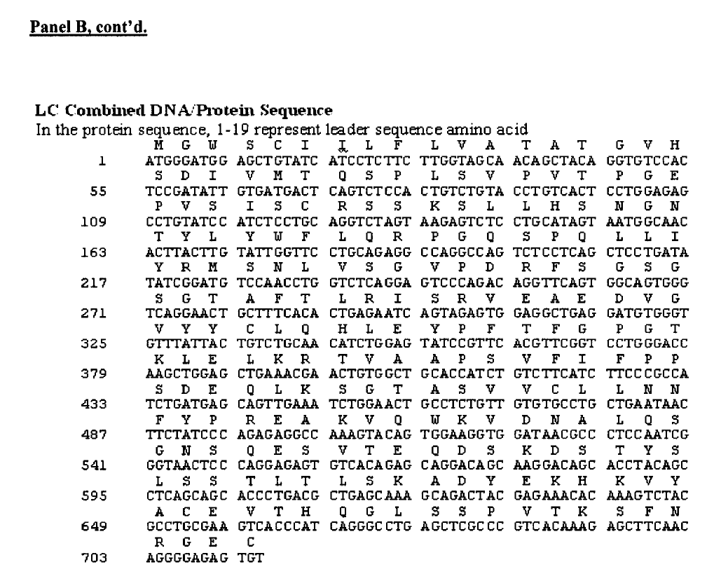

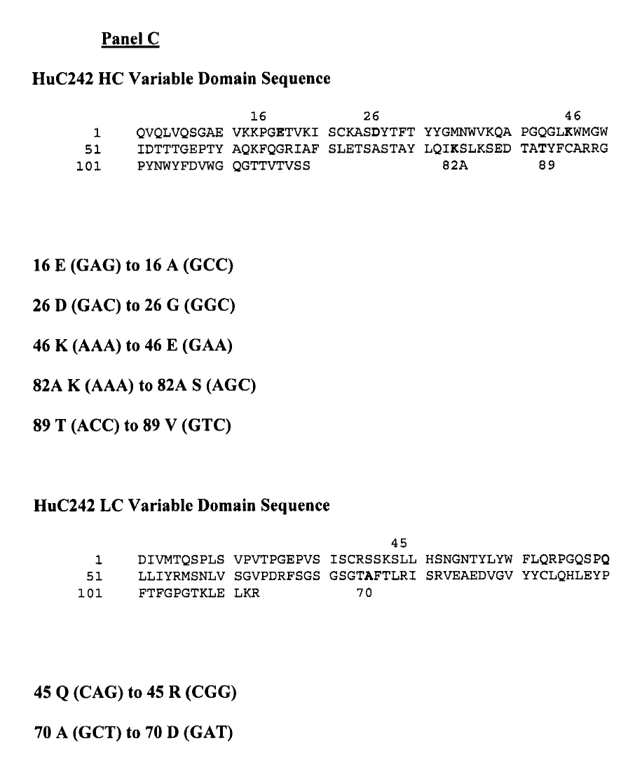

17. The nucleic acid of claim 16, wherein said at least one variation comprises a variation in a region of said sequence encoding a heavy chain variable region (FIG. 12) amino acid motif or a light chain variable region (FIG. 12) or both.

18. The nucleic acid of claim 17, wherein said at least one variation in said motif is selected from substitution of a codon encoding Gln (CAG) to Lys (AAA) or Arg (CGG), of a codon encoding Ala (GCT) to Asp (GAT), of a codon encoding Glu (GAG) to Ala (GCC), of a codon encoding Asp (GAC) to Gly (GGC), of a codon encoding Lys (AAA) to Glu (GAA); or of a codon encoding Thr (ACC) to Val (GTC).

19. The nucleic acid of claim 16, wherein said substitution of said codon occurs in a light or a heavy chain.

20. The nucleic acid of claim 16, wherein said light chain substitution is selected from:
Q45K/R Gln (CAG) to Lys (AAA) or Arg (CGG) or
A70D Ala (GCT) to Asp (GAT)

21. The nucleic acid of claim 16, wherein said heavy chain substitution is selected from
E16A Glu (GAG) to Ala (GCC);
D26G Asp (GAC) to Gly (GGC);
K46E Lys (AAA) to Glu (GAA); or
T89V Thr (ACC) to Val (GTC)

22. The nucleic acid of claims 16, wherein said sequence encodes a variant C242 gene product.

23. The nucleic acid of claim 22, wherein said gene product is an antibody.

24. A variant antibody or epitope binding fragment thereof, wherein said variant has one or more amino acid substitutions in a parent antibody having a variable region comprising a heavy chain of SEQ ID NO:1 [huC242] and a light chain of SEQ ID NO:2 [huC242] and said variant shows an improved synthesis when introduced in a single host cell as compared to said parent antibody.

25. The antibody of claim 24, wherein said substitution is at one or more positions selected from 45 and 70, in said SEQ ID NO:2 or 16, 26, 46, or 89 in said SEQ ID NO:1, said positions being determined by Kabat numbering scheme.

26. The antibody of claim 24, wherein said substitution is selected from the group consisting of Q45K/R, A70D, E16A, D26G, K46E, or T89V in the heavy chain, said positions being determined by Kabat numbering scheme.

27. A method for increasing production of a humanized antibody or an epitope binding fragment thereof in a host cell by sequence reengineering comprising:
a) aligning a collection of antibody variable region framework sequences from antibodies from the same genus or other close taxonomic classification as that to which the humanized antibody was derived belongs, wherein such alignment identifies amino acid residues most frequently found (consensus residues) at each position in the framework;
b) comparing the consensus residues with the corresponding residues in the humanized antibody variable region framework sequence;
c) identifying in the humanized antibody one or more non-consensus residues in the variable region framework sequence; and
d) substituting in the humanized antibody or a fragment thereof said one or more non-consensus residues with the consensus residue at the equivalent position to produce a variant antibody, wherein the variant antibody is produced in a cell at a higher yield as compared to the humanized antibody.
e) Optionally, one or more amino acids may be replaced with a non-consensus residue for biophysical considerations.

28. A method for increasing production of a parent antibody or a antigen binding fragment thereof in a host cell by sequence reengineering comprising:
a) aligning a collection of antibody variable region framework sequences from antibodies the same genus or close taxonomic classification as that to which the antibody from which the parent was derived belongs, wherein such alignment identifies amino acid residues most frequently found (consensus residues) at each position in the framework;
b) comparing the consensus residues with the corresponding residues in the parent antibody variable region framework sequence;
c) identifying in the parent antibody one or more non-consensus amino acid residues in the variable region framework sequence; and
d) substituting in the parent antibody or fragment thereof one or more non-consensus amino acid residues with the consensus residue at the equivalent position to produce a variant antibody, wherein the variant antibody is produced in the host cell at a higher yield as compared to the parent antibody.
e) Optionally, one or more amino acids may be replaced with a non-consensus residue for biophysical considerations.


SEQUENCE LISTING (16 sequences)

SEQ ID NO: 1 - protein, 119 aa, Artificial Sequence
  huC242 heavy chain
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 2 - protein, 112 aa, Artificial Sequence
  huC242 light chain
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 3 - protein, 132 aa, Artificial Sequence
  murine heavy chain consensus sequence
EVQLQQSGAELVKPGASVKLSCKASGYTFTSYWMHNSWVKQRPGKGLEWIGYINPKAGNGGTKYNEKFKGKATLTVDKSSSTAYMQLSSLTSEDSAVYYCARYYYYGGSYYYYYYYYYFDYWGQGTTVTVSS

SEQ ID NO: 4 - protein, 119 aa, Artificial Sequence
  murine light chain consensus sequence
DIVMTQSPASLSVSLGEKVTISCRASQSLLHSGNGNSYLHWYQQKPGQSPKLLIYDTSNLASGVPDRFSGSGSGTDFTLTISSVEAEDAATYYCQQWSSYPPSTHTPLTFGGGTKLEIK

SEQ ID NO: 5 - protein, 132 aa, Artificial Sequence
  huC242 light chain sequence
MGWSCIILFLVATATGVHSDIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKR

SEQ ID NO: 6 - protein, 132 aa, Artificial Sequence
  huMy96 light chain sequence
MGWSCIILFLVATATGVHSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 7 - protein, 122 aa, Artificial Sequence
  rB4 light chain sequence
MGWSCIILFLVATATGVHSEIVLTQSPAIMSASPGERVTMTCSASVNYMHWYQQKPGTSPRRWIYDTSKLASGVPARFSGSGSGTDYSLTISSMEPEDAATYYCHQRGSYTFGGGTKLEIKR

SEQ ID NO: 8 - protein, 132 aa, Artificial Sequence
  huEM164 light chain sequence
MGWSCIILFLVATATGVHSDVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 9 - protein, 132 aa, Artificial sequence
  huN901 light chain sequence
MGWSCIILFLVATATGVHSDVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKR

SEQ ID NO: 10 - protein, 121 aa, Artificial Sequence
  light chain consensus sequence
MGWSCIILFLVATATGVHSDIVMTQSPLSLPVSPGEPVSISCRSSQSIIHSNTYLWYQQKPGQSPRLLIYKVSNRSGVPDRFSGSGSGTDFTLRISRVEAEDLGIYYCQPTFGGTKLEIKR

SEQ ID NO: 11 - protein, 468 aa, Artificial Sequence
  huC242 heavy chain sequence
MGWSCIILFLVATATGVHSQVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1404 bp, Artificial Sequence
  huC242 heavy chain sequence
atgggatggagctgtatcatcctcttcttggtagcaacagctacaggtgtccactcccaggtccagttggtgcagtctggagccgaggtgaagaagcctggagagacagtcaagatctcctgcaaggcttctgattataccttcacatactatggaatgaactgggtgaagcaggctccgggacagggtctgaagtggatgggctggatcgacaccaccactggagagccaacatatgctcagaagtttcagggacggattgccttctctttggagacctctgccagcactgcctatctgcagatcaaaagcctgaaatctgaggacaccgctacatatttctgtgccagacgggggccttacaactggtactttgatgtctggggccaggggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 238 aa, Artificial Sequence
  huC242 light chain sequence
MGWSCIILFLVATATGVHSDIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 714 bp, Artificial Sequence
  huC242 light chain sequence
atgggatggagctgtatcatcctcttcttggtagcaacagctacaggtgtccactccgatattgtgatgactcagtctccactgtctgtacctgtcactcctggagagcctgtatccatctcctgcaggtctagtaagagtctcctgcatagtaatggcaacacttacttgtattggttcctgcagaggccaggccagtctcctcagctcctgatatatcggatgtccaacctggtctcaggagtcccagacaggttcagtggcagtgggtcaggaactgctttcacactgagaatcagtagagtggaggctgaggatgtgggtgtttattactgtctgcaacatctggagtatccgttcacgttcggtcctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 119 aa, Artificial Sequence; identical to SEQ ID NO: 1
  huC242 heavy chain variable domain sequence

SEQ ID NO: 16 - protein, 113 aa, Artificial Sequence
  huC242 light chain variable domain sequence
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKR